SEQUENCE LISTING (16 sequences)

SEQ ID NO: 1 - protein, 176 aa, Homo sapiens
MSAGGASVPPPPNPAVSFPPPRVTLPAGPDILRTYSGAFVCLEILFGGLVWILVASSNVPLPLLQGWVMFVSVTAFFFSLLFLGMFLSGMVAQIDANWNFLDFAYHFTVFVFYFGAFLLEAAATSLHDLHCNTTITGQPLLSDNQYNINVAASIFAFMTTACYGCSLGLALRRWRP

SEQ ID NO: 2 - DNA, 2808 bp, Artificial Sequence
  Description of Artificial Sequencenucleotide sequence of the human MAL2 cDNA
ggcggcggcggcaggagcccgggaggcggaggcgggaggcggcggcggcgcgcggagacgcagcagcggcagcggcagcatgtcggccggcggagcgtcagtcccgccgcccccgaaccccgccgtgtccttcccgccgccccgggtcaccctgcccgccggccccgacatcctgcggacctactcgggcgccttcgtctgcctggagattctgttcgggggtcttgtctggattttggttgcctcctccaatgttcctctacctctactacaaggatgggtcatgtttgtgtccgtgacagcgtttttcttttcgctcctctttctgggcatgttcctctctggcatggtggctcaaattgatgctaactggaacttcctggattttgcctaccattttacagtatttgtcttctattttggagcctttttattggaagcagcagccacatccctgcatgatttgcattgcaatacaaccataaccgggcagccactcctgagtgataaccagtataacataaacgtagcagcctcaatttttgcctttatgacgacagcttgttatggttgcagtttgggtctggctttacgaagatggcgaccgtaacactccttagaaactggcagtcgtatgttagtttcacttgtctactttatatgtctgatcaatttggataccattttgtccagatgcaaaaacattccaaaagtaatgtgtttagtagagagagactctaagctcaagttctggtttatttcatggatggaatgttaattttattatgatattaaagaaatggccttttattttacatctctcccctttttccctttccccctttattttcctccttttctttctgaaagtttccttttatgtccataaaatacaaatatattgttcataaaaaattagtatcccttttgtttggttgctgagtcacctgaaccttaattttaattggtaattacagcccctaaaaaaaacacatttcaaataggcttcccactaaactctatattttagtgtaaaccaggaattggcacactttttttagaatgggccagatggtaaatatttatgcttcacggtccatacagtctctgtcacaactattcagttctgctagtatagcgtgaaagcagctatacacaatacagaaatgaatgagtgtggttatgttctaataaaacttatttataaaaacaaggggaggctgggtttagcctgtgggccatagtttgtcaaccactggtgtaaaaccttagttatatatgatctgcattttcttgaactgatcattgaaaacttataaacctaacagaaaagccacataatatttagtgtcattatgcaataatcacattgcctttgtgttaatagtcaaatacttacctttggagaatacttacctttggaggaatgtataaaatttctcaggcagagtcctggatataggaaaaagtaatttatgaagtaaacttcagttgcttaatcaaactaatgatagtctaacaactgagcaagatcctcatctgagagtgcttaaaatgggatccccagagaccattaaccaatactggaactggtatctagctactgatgtcttactttgagtttatttatgcttcagaatacagttgtttgccctgtgcatgaatatacccatatttgtgtgtggatatgtgaagcttttccaaatagagctctcagaagaattaagtttttacttctaattattttgcattactttgagttaaatttgaatagagtattaaatataaagttgtagattcttatgtgtttttgtattagcccagacatctgtaatgtttttgcactggtgacagacaaaatctgttttaaaatcatatccagcacaaaaactatttctggctgaatagcacagaaaagtattttaacctacctgtagagatcctcgtcatggaaaggtgccaaactgttttgaatggaaggacaagtaagagtgaggccacagttcccaccacacgagggcttttgtattgttctactttttcagccctttactttctggctgaagcatccccttggagtgccatgtataagttgggctattagagttcatggaacatagaacaaccatgaatgagtggcatgatccgtgcttaatgatcaagtgttacttatctaataatcctctagaaagaaccctgttagatcttggtttgtgataaaaatataaagacagaagacatgaggaaaaacaaaaggtttgaggaaatcaggcatatgactttatacttaacatcagatcttttctataatatcctactactttggttttcctagctccataccacacacctaaacctgtattatgaattacatattacaaagtcataaatgtgccatatggatatacagtacattctagttggaatcgtttactctgctagaatttaggtgtgagattttttgtttcccaggtatagcaggcttatgtttggtggcattaaattggtttctttaaaatgctttggtggcacttttgtaaacagattgcttctagattgttacaaaccaagcctaagacacatctgtgaatacttagatttgtagcttaatcacattctagacttgtgagttgaatgacaaagcagttgaacaaaaattatggcatttaagaatttaacatgtcttagctgtaaaaatgagaaagtgttggttggttttaaaatctggtaactccatgatgaaaagaaatttattttatacgtgttatgtctctaataaagtattcatttgataaaaaaaaaaaaaaa

SEQ ID NO: 3 - DNA, 270 bp, Artificial Sequence
  Description of Artificial Sequencenucleotide sequence of the 270 bp MAL2 cDNA fragment
tggtggcacttttgtaaacagattgcttctagattgttacaaaccaagcctaagacacatctgtgaatacttagatttgtagcttaatcacattctagacttgtgagttgaatgacaaagcagttgaacaaaaattatggcatttaagaatttaacatgtcttagctgtaaaaatgagaaagtgttggttggttttaaaatctggtaactccatgatggaaagaaatttattttatacgtgttatgtctctaataaagtattcatttgat

SEQ ID NO: 4 - DNA, 531 bp, Artificial Sequence
  Description of Artificial Sequencecoding sequence of the human MAL2 gene
atgtcggccggcggagcgtcagtcccgccgcccccgaaccccgccgtgtccttcccgccgccccgggtcaccctgcccgccggccccgacatcctgcggacctactcgggcgccttcgtctgcctggagattctgttcgggggtcttgtctggattttggttgcctcctccaatgttcctctacctctactacaaggatgggtcatgtttgtgtccgtgacagcgtttttcttttcgctcctctttctgggcatgttcctctctggcatggtggctcaaattgatgctaactggaacttcctggattttgcctaccattttacagtatttgtcttctattttggagcctttttattggaagcagcagccacatccctgcatgatttgcattgcaatacaaccataaccgggcagccactcctgagtgataaccagtataacataaacgtagcagcctcaatttttgcctttatgacgacagcttgttatggttgcagtttgggtctggctttacgaagatggcgaccgtaa

SEQ ID NO: 5 - DNA, 23 bp, Artificial Sequence
  Description of Artificial Sequenceprimer for the human MAL2 gene
acctgtagagatcctcgtcatgg

SEQ ID NO: 6 - DNA, 23 bp, Artificial Sequence
  Description of Artificial Sequenceprimer for the human MAL2 gene
tggcctcactcttacttgtcctt

SEQ ID NO: 7 - DNA, 20 bp, Artificial Sequence
  Description of Artificial Sequenceprimer for the human cyclophilin B gene
actgaagcactacgggcctg

SEQ ID NO: 8 - DNA, 19 bp, Artificial Sequence
  Description of Artificial Sequenceprimer for the human cyclophilin B gene
agccgttggtgtctttgcc

SEQ ID NO: 9 - DNA, 20 bp, Artificial Sequence
  Description of Artificial Sequenceprimer for the human ribosomal protein S9 gene
ggtcaaatttaccctggcca

SEQ ID NO: 10 - DNA, 22 bp, Artificial Sequence
  Description of Artificial Sequenceprimer for the human ribosomal protein S9 gene
tctcatcaagcgtcagcagttc

SEQ ID NO: 11 - DNA, 19 bp, Artificial Sequence
  Description of Artificial Sequenceprimer for the human beta-actin gene
tggaacggtgaaggtgaca

SEQ ID NO: 12 - DNA, 19 bp, Artificial Sequence
  Description of Artificial Sequenceprimer for the human beta-actin gene
ggcaagggacttcctgtaa

SEQ ID NO: 13 - DNA, 20 bp, Artificial Sequence
  Description of Artificial Sequenceprimer for the human GAPDH gene
cgtcatgggtgtgaaccatg

SEQ ID NO: 14 - DNA, 21 bp, Artificial Sequence
  Description of Artificial Sequenceprimer for the human GAPDH gene
gctaagcagttggtggtgcag

SEQ ID NO: 15 - DNA, 21 bp, Artificial Sequence
  Description of Artificial Sequenceprimer for the human transferrin receptor TRR gene
gtcgctggtcagttcgtgatt

SEQ ID NO: 16 - DNA, 23 bp, Artificial Sequence
  Description of Artificial Sequenceprimer for the human transferrin receptor TRR gene
agcagttggctgttgtacctctc

(19) United States
(12) Patent Application Publication
(10) Pub. No.: US 2007/0166718 A1
(43) Pub. Date: Jul. 19, 2007

(54) Diagnostic and therapeutic use of mal2 gene and protein for neurodegenerative diseases

(75) Inventors: Heinz Von Der Kammer, Hamburg (DE); Johannes Pohlner, Hamburg (DE)

Correspondence Address: STERNE, KESSLER, GOLDSTEIN & FOX P.L.L.C., 1100 NEW YORK AVENUE, N.W., WASHINGTON, DC, 20005, US

(73) Assignee: EVOTEC NEUROSCIENCES GMBH, HAMBURG (DE)

(21) Appl. No.: 10/591,678

(22) PCT Filed: Feb. 28, 2005
(86) PCT No.: PCT/EP05/50850

§ 371 Date: Sep. 1, 2006

Related U.S. Application Data
(60) Provisional application No. 60/549,147, filed on Mar. 3, 2004.

(51) Int. Cl. C12Q 1/68; G01N 33/567

(52) U.S. Cl. 435/6; 435/7.2

(57) ABSTRACT

The present invention discloses the differential expression of the gene coding for MAL2 protein in specific brain regions of Alzheimer's disease patients. Based on this finding, the invention provides a method for diagnosing or prognosticating Alzheimer's disease in a subject, or for determining whether a subject is at increased risk of developing Alzheimer's disease. Furthermore, this invention provides therapeutic and prophylactic methods for treating or preventing Alzheimer's disease and related neurodegenerative disorders using the MAL2 gene and its corresponding gene products. A method of screening for modulating agents of neurodegenerative diseases is also disclosed.

The present invention relates to methods of diagnosing, prognosticating and monitoring the progression of neurodegenerative diseases in a subject. Furthermore, methods of therapy control and screening for modulating agents of neurodegenerative diseases are provided. The invention also discloses pharmaceutical compositions, kits, and recombinant animal models.

Neurodegenerative diseases, in particular Alzheimer's disease (AD), have a strongly debilitating impact on a patient's life. Furthermore, these diseases constitute an enormous health, social, and economic burden. AD is the most common neurodegenerative disease, accounting for about 70% of all dementia cases, and it is probably the most devastating age-related neurodegenerative condition affecting about 10% of the population over 65 years of age and up to 45% over age 85 (for a recent review see Vickers et al., Progress in Neurobiology 2000, 60: 139-165). Presently, this amounts to an estimated 12 million cases in the US, Europe, and Japan. This situation will inevitably worsen with the demographic increase in the number of old people (“aging of the baby boomers”) in developed countries. The neuropathological hallmarks that occur in the brains of individuals with AD are senile plaques, composed of amyloid-β protein, and profound cytoskeletal changes coinciding with the appearance of abnormal filamentous structures and the formation of neurofibrillary tangles.

The amyloid-β protein evolves from the cleavage of the amyloid precursor protein (APP) by different kinds of proteases. The cleavage by the β/γ-secretase leads to the formation of Aβ peptides of different lengths, typically a short more soluble and slow aggregating peptide consisting of 40 amino acids and a longer 42 amino acid peptide, which rapidly aggregates outside the cells, forming the characteristic amyloid plaques (Selkoe, Physiological Rev 2001, 81:741-66; Greenfield et al., Frontiers Bioscience 2000, 5: D72-83). They are primarily found in the cerebral cortex and hippocampus. The generation of toxic AP deposits in the brain starts very early in the course of AD, and it is discussed to be a key player for the subsequent destructive processes leading to AD pathology. The other pathological hallmarks of AD are neurofibrillary tangles (NFTs) and abnormal neurites, described as neuropil threads (Braak and Braak, Acta Neuropathol 1991, 82: 239-259). NFTs emerge inside neurons and consist of chemically altered tau, which forms paired helical filaments twisted around each other. The appearance of neurofibrillary tangles and their increasing number correlates well with the clinical severity of AD (Schmitt et al., Neurology 2000, 55: 370-376).

AD is a progressive disease that is associated with early deficits in memory formation and ultimately leads to the complete erosion of higher cognitive function. The cognitive disturbances include among other things memory impairment, aphasia, agnosia and the loss of executive functioning. A characteristic feature of the pathogenesis of AD is the selective vulnerability of particular brain regions and subpopulations of nerve cells to the degenerative process. Specifically, the temporal lobe region and the hippocampus are affected early and more severely during the progression of the disease. On the other hand, neurons within the frontal cortex, occipital cortex, and the cerebellum remain largely intact and are protected from neurodegeneration (Terry et al., Annals of Neurology 1981, 10: 184-92). The age of onset of AD may vary within a range of 50 years, with early-onset AD occurring in people younger than 65 years of age, and late-onset of AD occurring in those older than 65 years.

Currently, there is no cure for AD, nor is there an effective treatment to halt the progression of AD or even to diagnose AD ante-mortem with high probability. Several risk factors have been identified that predispose an individual to develop AD, among them most prominently the epsilon 4 allele of the three different existing alleles (epsilon 2, 3, and 4) of the apolipoprotein E gene (ApoE) (Strittmatter et al., Proc Natl Acad Sci USA 1993, 90: 1977-81; Roses, Ann NY Acad Sci 1998, 855: 738-43). Although there are rare examples of early-onset AD which have been attributed to genetic defects in the genes for amyloid precursor protein (APP) on chromosome 21, presenilin-1 on chromosome 14, and presenilin-2 on chromosome 1, the prevalent form of late-onset sporadic AD is of hitherto unknown etiologic origin. The late onset and complex pathogenesis of neurodegenerative disorders pose a formidable challenge to the development of therapeutic and diagnostic agents. It is crucial to expand the pool of potential drug targets and diagnostic markers. It is therefore an object of the present invention to provide insight into the pathogenesis of neurological diseases and to provide methods, materials, agents, compositions, and animal models which are suited inter alia for the diagnosis and development of a treatment of these diseases. This object has been solved by the features of the independent claims. The subclaims define preferred embodiments of the present invention.

Myelin and lymphocyte proteolipid (MAL) is the founder member of a synonymous family of proteins with structural and biochemical similarities (Perez et al., Biochem Biophys Res Commun 1997, 232:618-621). MAL is a nonglycosylated integral membrane protein that exclusively resides in rafts, containing four hydrophobic transmembrane (TM1-4) segments, with cytoplasmic N- and C-termini, forming a so-called “MARVEL” domain (MAL and Related proteins for Vesicle trafficking and membrane Link), a conserved domain involved in membrane apposition events. MAL plays an essential role in the direct-route transport of proteins with apical destination. As an itinerant protein, MAL cyclically shuttles between the trans-Golgi network, the plasma membrane and the endosomes. MAL is required for the correct delivery of apical cargo (both membrane bound and secretory proteins) within polarized cells (e.g. epithelia, oligodendrocytes, Schwann cells), and it is a key element in the formation, sorting and transport of vesicles, and in cholesterol-rich membrane apposition events, including the organization and maintenance of membrane microdomains/lipid rafts (Puertollano and Alonso, Mol Biol Cell 1999, 10:3435-3447; Martin-Belmonte et al., J Biol Chem 2001, 276:49337-49342; Sanchez-Pulido et al., Trends Biochem Sci 2002, 27:599-601; Erne et al., J Neurochem 2002, 82:550-562).

Not only epithelial cells, but also myelin forming glia cells in the central (CNS) and peripheral nervous system (PNS), oligodendrocytes and Schwann cells, are polarized cells with distinct transport and sorting pathways. A proposed model of sphingolipid-cholesterol raft dependent organization and control of transport and sorting may therefore also apply to myelin. MAL is expressed by oligodendrocytes and Schwann cells as a component of central and peripheral myelin, where it is selectively enriched, together with CD59, in detergent-insoluble glycolipid enriched membrane microdomains (DIGs). In the CNS, MAL is involved in late steps of myelin sheath formation and myelin compaction, whereas in the PNS it plays a role in the terminal differentiation of maturating Schwann cells and in the onset of myelination (Erne et al., J Neurochem 2002, 82:550-562; Frank et al., J Neurochem 2000, 75:1927-1939; Frank et al., J Neurochem 1999, 73:587-597).

MAL2 has recently been identified as a novel member of the MAL family, sharing 36% sequence identity with MAL at the protein level (Wilson et al., Genomics 2001, 76:81-88; Genbank data base accession numbers: genomic DNA contig AC009514, cDNA AY007723, protein Q969L2). The MAL2 gene maps to chromosome 8q24.12 and it comprises 4 exons. Like MAL, MAL2 is an integral membrane protein whose structure is based on the characteristic 4 transmembrane-helix MARVEL domain with cytoplasmic N- and C-termini. It also contains the characteristic MAL-like sequence motif (Q/F)GWVM(F/Y)V in TM2 (Gln65-Val71). Furthermore, the C-terminal LRRW sequence motif of MAL2 (aa 171-174) is similar to the MAL motif LIRW (aa 146-149), which has been shown to be a minimal requirement for the targeting to glycolipid enriched membrane microdomains. Despite these similarities, MAL2 differs from MAL and other MAL-like proteins in some other aspects: it has a longer N-terminal domain (34 instead of 15-22 aa) with a higher proline content (29% versus 7-19%) and an FPPP sequence (aa 18-21) resembling an FPPPP recognition motif for EVH1 (enabled, VASP, homology 1) domains; and it possesses an additional 10-aa insertion (Ser125-Thr124) predicting a larger loop of 31-aa between TM3 and TM4 (luminal), with a single N-glycosylation site (Asn132). Whereas MAL is not glycosylated, Western blot analysis of MAL2 revealed a distinct band at 19 kDa and an endogycosylase H sensitive smear at ˜30-40 kDa, reflecting both unglycosylated and glycosylated MAL2, respectively, indicating that MAL2 is partly N-glycosylated (Wilson et al., Genomics 2001, 76:81-88; DeMarco et al., J Cell Biol 2002, 159:37-44; Sanchez-Pulido et al., Trends Biochem Sci 2002, 27:599-601; Magyar et al., Gene 1997, 189:269-275). Northern blot analyses of human tissue mRNA extracts revealed a major 2.8 kb MAL2 transcript with highest signal intensity in the kidney and in mammary carcinoma, moderate levels in brain, liver and lung, weak expression in heart, placenta, colon (without mucosa) and small intestine. A minor 1.2 kb transcript produced a moderate signal in mammary carcinoma and weak signals in kidney and liver. An endogenous 2.8 kb transcript was detected in HepG2, Caco-2 (both human) and MDCK (canine) cell lines, but not in Jurkat, HPB-ALL, A498, HeLa and K-562 (all human) or COS-7 (simian) cells. The 1.2 kb signal has been proposed to reflect the existence of a 3′-UTR truncated MAL2 transcript due to an alternative (earlier) polyadenylation (AATAAA) signal within the 3′-UTR (nt 1190-1195 of AY007723) (DeMarco et al., J Cell Biol 2002, 159:37-44; Wilson et al., Genomics 2001, 76:81-88). DeMarco et al. (J Cell Biol 2002, 159:37-44) have shown that antisense oligonucleotide mediated depletion of endogenous MAL2 drastically blocked apically targeted transport of both exogenous and endogenous transcytosing molecules at perinuclear endosomes. MAL2 depletion did not affect the internalization of these molecules but caused their accumulation in perinuclear endosome elements that were accessible to transferrin. From their data DeMarco et al. conclude that both MAL and MAL2 are essential and functionally distinct members of the machinery of polarized transport, in that MAL plays a key role in the direct apical transport pathway, and MAL2 is required for the indirect transcytotic route.

The singular forms “a”, “an”, and “the” as used herein and in the claims include plural reference unless the context dictates otherwise. For example, “a cell” means as well a plurality of cells, and so forth. The term “and/or” as used in the present specification and in the claims implies that the phrases before and after this term are to be considered either as alternatives or in combination. For instance, the wording “determination of a level and/or an activity” means that either only a level, or only an activity, or both a level and an activity are determined. The term “level” as used herein is meant to comprise a gage of, or a measure of the amount of, or a concentration of a transcription product, for instance an mRNA, or a translation product, for instance a protein or polypeptide. The term “activity” as used herein shall be understood as a measure for the ability of a transcription product or a translation product to produce a biological effect or a measure for a level of biologically active molecules. The term “activity” also refers to enzymatic activity. The terms “level” and/or “activity” as used herein further refer to gene expression levels or gene activity. Gene expression can be defined as the utilization of the information contained in a gene by transcription and translation leading to the production of a gene product. “Dysregulation” shall mean an upregulation or downregulation of gene expression. A gene product comprises either RNA or protein and is the result of expression of a gene. The amount of a gene product can be used to measure how active a gene is. The term “gene” as used in the present specification and in the claims comprises both coding regions (exons) as well as non-coding regions (e.g. non-coding regulatory elements such as promoters or enhancers, introns, leader and trailer sequences). The term “ORF” is an acronym for “open reading frame” and refers to a nucleic acid sequence that does not possess a stop codon in at least one reading frame and therefore can potentially be translated into a sequence of amino acids. “Regulatory elements” shall comprise inducible and non-inducible promoters, enhancers, operators, and other elements that drive and regulate gene expression. The term “fragment” as used herein is meant to comprise e.g. an alternatively spliced, or truncated, or otherwise cleaved transcription product or translation product. The term “derivative” as used herein refers to a mutant, or an RNA-edited, or a chemically modified, or otherwise altered transcription product, or to a mutant, or chemically modified, or otherwise altered translation product. For the purpose of clarity, a derivative transcript, for instance, refers to a transcript having alterations in the nucleic acid sequence such as single or multiple nucleotide deletions, insertions, or exchanges. A derivative translation product, for instance, may be generated by processes such as altered phosphorylation, or glycosylation, or acetylation, or lipidation, or by altered signal peptide cleavage or other types of maturation cleavage. These processes may occur post-translationally. Preferably, a “modulator” is capable of changing or altering the biological activity of a transcription product or a translation product of a gene. Said modulation, for instance, may be an increase or a decrease in the biological activity and/or pharmacological activity, in enzyme activity, a change in binding characteristics, or any other change or alteration in the biological, functional, or immunological properties of said translation product of a gene. A “modulator” refers to a molecule which has the capacity to either enhance or inhibit, thus to “modulate” a functional property of an ion channel subunit or an ion channel, to “modulate” binding, antagonization, repression, blocking, neutralization or sequestration of an ion channel or ion channel subunit and to “modulate” activation, agonization and upregulation. “Modulation” will be also used to refer to the capacity to affect the biological activity of a cell. The terms “modulator”, “agent”, “reagent”, or “compound” refer to any substance, chemical, composition, or extract that have a positive or negative biological effect on a cell, tissue, body fluid, or within the context of any biological system, or any assay system examined. They can be agonists, antagonists, partial agonists or inverse agonists of a target. They may be nucleic acids, natural or synthetic peptides or protein complexes, or fusion proteins. They may also be antibodies, organic or anorganic molecules or compositions, small molecules, drugs and any combinations of any of said agents above. They may be used for testing, for diagnostic or for therapeutic purposes. Such modulators, agents, reagents or compounds can be factors present in cell culture media, or sera used for cell culturing, factors such as trophic factors. The terms “oligonucleotide primer” or “primer” refer to short nucleic acid sequences which can anneal to a given target polynucleotide by hybridization of the complementary base pairs and can be extended by a polymerase. They may be chosen to be specific to a particular sequence or they may be randomly selected, e.g. they will prime all possible sequences in a mix. The length of primers used herein may vary from 10 nucleotides to 80 nucleotides. “Probes” are short nucleic acid sequences of the nucleic acid sequences described and disclosed herein or sequences complementary therewith. They may comprise full length sequences, or fragments, derivatives, isoforms, or variants of a given sequence. The identification of hybridization complexes between a “probe” and an assayed sample allows the detection of the presence of other similar sequences within that sample. As used herein, “homolog or homology” is a term used in the art to describe the relatedness of a nucleotide or peptide sequence to another nucleotide or peptide sequence, which is determined by the degree of identity and/or similarity between said sequences compared. In the art, the terms “identity” and “similarity” mean the degree of polypeptide or polynucleotide sequence relatedness which are determined by matching a query sequence and other sequences of preferably the same type (nucleic acid or protein sequence) with each other. Preferred computer program methods to calculate and determine “identity” and “similarity” include, but are not limited to GCG BLAST (Basic Local Alignment Search Tool) (Altschul et al., J. Mol. Biol. 1990, 215: 403-410; Altschul et al., Nucleic Acids Res. 1997, 25: 3389-3402; Devereux et al., Nucleic Acids Res. 1984, 12: 387), BLASTN 2.0 (Gish W., http://blast.wustl.edu, 1996-2002), FASTA (Pearson and Lipman, Proc. Natl. Acad. Sci. USA 1988, 85: 2444-2448), and GCG GelMerge which determines and aligns a pair of contigs with the longest overlap (Wilbur and Lipman, SIAM J. Appl. Math. 1984, 44: 557-567; Needleman and Wunsch, J. Mol. Biol. 1970, 48: 443-453). The term “variant” as used herein refers to any polypeptide or protein, in reference to polypeptides and proteins disclosed in the present invention, in which one or more amino acids are added and/or substituted and/or deleted and/or inserted at the N-terminus, and/or the C-terminus, and/or within the native amino acid sequences of the native polypeptides or proteins of the present invention, but retains its essential properties. Furthermore, the term “variant” shall include any shorter or longer version of a polypeptide or protein. “Variants” shall also comprise a sequence that has at least about 80% sequence identity, more preferably at least about 90% sequence identity, and most preferably at least about 95% sequence identity with the amino acid sequences of MAL2 protein, SEQ ID NO: 1. “Variants” include, for example, proteins with conservative amino acid substitutions in highly conservative regions. “Proteins and polypeptides” of the present invention include variants, fragments and chemical derivatives of the protein comprising the amino acid sequences of MAL2 protein, SEQ ID NO: 1. They can include proteins and polypeptides which can be isolated from nature or be produced by recombinant and/or synthetic means. Native proteins or polypeptides refer to naturally-occurring truncated or secreted forms, naturally occurring variant forms (e.g. splice-variants) and naturally occurring allelic variants. The term “isolated” as used herein is considered to refer to molecules or substances which have been changed and/or that are removed from their natural environment, i.e. isolated from a cell or from a living organism in which they normally occur, and that are separated or essentially purified from the coexisting components with which they are found to be associated in nature, it is also said that they are “non-native”. This notion further means that the sequences encoding such molecules can be linked by the hand of man to polynucleotides to which they are not linked in their natural state and such molecules can be produced by recombinant and/or synthetic means (non-native). Even if for said purposes those sequences may be introduced into living or non-living organisms by methods known to those skilled in the art, and even if those sequences are still present in said organisms, they are still considered to be isolated, to be non-native. In the present invention, the terms “risk”, “susceptibility”, and “predisposition” are tantamount and are used with respect to the probability of developing a neurodegenerative disease, preferably Alzheimer's disease.

The term “AD” shall mean Alzheimer's disease. “AD-type neuropathology” as used herein refers to neuropathological, neurophysiological, histopathological and clinical hallmarks as described in the instant invention and as commonly known from state-of-the-art literature (see: Iqbal, Swaab, Winblad and Wisniewski, Alzheimer's Disease and Related Disorders (Etiology, Pathogenesis and Therapeutics), Wiley & Sons, New York, Weinheim, Toronto, 1999; Scinto and Daffner, Early Diagnosis of Alzheimer's Disease, Humana Press, Totowa, N.J., 2000; Mayeux and Christen, Epidemiology of Alzheimer's Disease: From Gene to Prevention, Springer Press, Berlin, Heidelberg, N.Y., 1999; Younkin, Tanzi and Christen, Presenilins and Alzheimer's Disease, Springer Press, Berlin, Heidelberg, N.Y., 1998). The term “Braak stage” or “Braak staging” refers to the classification of brains according to the criteria proposed by Braak and Braak (Braak and Braak, Acta Neuropathology 1991, 82: 239-259). On the basis of the distribution of neurofibrillary tangles and neuropil threads, the neuropathologic progression of AD is divided into six stages (stage 0 to 6). In the instant invention Braak stages 0 to 2 represent healthy control persons (“controls”), and Braak stages 4 to 6 represent persons suffering from Alzheimer's disease (“AD patients”). The values obtained from said “controls” are the “reference values” representing a “known health status” and the values obtained from said “AD patients” are the “reference values” representing a “known disease status”. Braak stage 3 (middle Braak stage) may represent either a healthy control persons or an AD patient. The higher the Braak stage the more likely is the possibility to display the symptoms of AD. For a neuropathological assessment, i.e. an estimation of the probability that pathological changes of AD are the underlying cause of dementia, a recommendation is given by Braak H. (www alzforum.org).

Neurodegenerative diseases or disorders according to the present invention comprise Alzheimer's disease, Parkinson's disease, Huntington's disease, amyotrophic lateral sclerosis, Pick's disease, fronto-temporal dementia, progressive nuclear palsy, corticobasal degeneration, cerebro-vascular dementia, multiple system atrophy, argyrophilic grain dementia and other tauopathies, and mild-cognitive impairment. Further conditions involving neurodegenerative processes are, for instance, ischemic stroke, age-related macular degeneration, narcolepsy, motor neuron diseases, prion diseases, traumatic nerve injury and repair, and multiple sclerosis.

The present invention discloses the detection, identification and the differential regulation, a dysregulation of a gene of the Myelin and lymphocyte proteolipid (MAL) gene family coding for a member of the MAL family of proteins, the Myelin and lymphocyte proteolipid (MAL) protein MAL2, in samples of specific brain regions of Alzheimer's disease patients in comparison to the respective samples of age-matched control persons. The present invention discloses that the gene expression for MAL2 is varied, is dysregulated within different regions of AD-affected brains, in that MAL2 mRNA levels are lowered, are down-regulated in the temporal cortex and/or the hippocampus as compared to the frontal cortex, or are upregulated in the frontal cortex as compared to the temporal cortex and/or the hippocampus. Further, the present invention discloses that the MAL2 expression differs between the frontal cortex and the temporal cortex and/or the hippocampus of healthy age-matched control subjects compared to the frontal cortex and the temporal cortex and/or the hippocampus of AD patients. No such dysregulation is observed within samples of different brain regions obtained from age-matched, healthy controls. MAL2 is lowered in the temporal cortex and in the frontal cortex of AD-patients compared to the temporal cortex and frontal cortex of controls. This dysregulation presumably relates to a pathologic alteration of MAL2 signaling in AD-affected brains. To date, no experiments have been described that demonstrate a relationship between the dysregulation of MAL2 gene expression and the pathology of neurodegenerative diseases, in particular AD. Likewise, no mutations in the MAL2 gene have been described to be associated with said diseases. Linking the MAL2 gene to such diseases offers new ways, inter alia, for the diagnosis and treatment of said diseases.

The present invention discloses a dysregulation of a gene coding for MAL2 in specific brain regions of AD patients. Neurons within the inferior temporal lobe, the entorhinal cortex, the hippocampus, and the amygdala are subject to degenerative processes in AD (Terry et al., Annals of Neurology 1981, 10:184-192). These brain regions are mostly involved in the processing of learning and memory functions and display a selective vulnerability to neuronal loss and degeneration in AD. In contrast, neurons within the frontal cortex, the occipital cortex, and the cerebellum remain largely intact and preserved from neurodegenerative processes. Therefor, brain tissues from the frontal cortex (F), the temporal cortex (T), and the hippocampus (H) of AD patients and of healthy, age-matched control individuals, respectively, were used for the herein disclosed examples. Consequently, the MAL2 gene and its corresponding transcription and/or translation products have a causative role in the regional selective neuronal degeneration typically observed in AD. Alternatively, the gene coding for MAL2 protein and its products may confer neuroprotective functions to nerve cells. Based on these disclosures, the present invention has utility for the diagnostic evaluation and prognosis as well as for the identification of a predisposition to a neurodegenerative disease, in particular AD. Furthermore, the present invention provides methods for the diagnostic monitoring of patients undergoing treatment for such a disease.

In one aspect, the invention features a method of diagnosing or prognosticating a neurodegenerative disease in a subject, or determining whether a subject is at increased risk of developing said disease. The method comprises: determining a level, or an activity, or both said level and said activity of (i) a transcription product of the gene coding for MAL2 protein, and/or of (ii) a translation product of the gene coding for MAL2 protein, and/or of (iii) a fragment, or derivative, or variant of said transcription or translation product in a sample obtained from said subject and comparing said level, and/or said activity of said transcription product and/or said translation product to a reference value representing a known disease status and/or to a reference value representing a known health status (healthy control), and said level and/or said activity is varied, is altered compared to a reference value representing a known health status, and/or is similar or equal to a reference value representing a known disease status, thereby diagnosing or prognosticating said neurodegenerative disease in said subject, or determining whether said subject is at increased risk of developing said neurodegenerative disease. The wording “in a subject” refers to results of the methods disclosed as far as they relate to a disease afflicting a subject, that is to say, said disease being “in” a subject.

The invention also relates to the construction and the use of primers and probes which are unique to the nucleic acid sequences, or fragments, or variants thereof, as disclosed in the present invention. The oligonucleotide primers and/or probes can be labeled specifically with fluorescent, bioluminescent, magnetic, or radioactive substances. The invention further relates to the detection and the production of said nucleic acid sequences, or fragments and variants thereof, using said specific oligonucleotide primers in appropriate combinations. PCR-analysis, a method well known to those skilled in the art, can be performed with said primer combinations to amplify said gene specific nucleic acid sequences from a sample containing nucleic acids. Such sample may be derived either from healthy or diseased subjects. Whether an amplification results in a specific nucleic acid product or not, and whether a fragment of different length can be obtained or not, may be indicative for a neurodegenerative disease, in particular Alzheimer's disease. Thus, the invention provides nucleic acid sequences, oligonucleotide primers, and probes of at least 10 bases in length up to the entire coding and gene sequences, useful for the detection of gene mutations and single nucleotide polymorphisms in a given sample comprising nucleic acid sequences to be examined, which may be associated with neurodegenerative diseases, in particular Alzheimer's disease. This feature has utility for developing rapid DNA-based diagnostic tests, preferably also in the format of a kit. Primers for MAL2 are exemplarily described in Example (iv).

In a further aspect, the invention features a method of monitoring the progression of a neurodegenerative disease in a subject. A level, or an activity, or both said level and said activity, of (i) a transcription product of the gene coding for MAL2 protein, and/or of (ii) a translation product of the gene coding for MAL2 protein, and/or of (iii) a fragment, or derivative, or variant of said transcription or translation product in a sample from said subject is determined. Said level and/or said activity is compared to a reference value representing a known disease or health status. Thereby, the progression of said neurodegenerative disease in said subject is monitored.

In still a further aspect, the invention features a method of evaluating a treatment for a neurodegenerative disease, comprising determining a level, or an activity, or both said level and said activity of (i) a transcription product of the gene coding for MAL2 protein, and/or of (ii) a translation product of the gene coding for MAL2 protein, and/or of (iii) a fragment, or derivative, or variant of said transcription or translation product in a sample obtained from a subject being treated for said disease. Said level, or said activity, or both said level and said activity are compared to a reference value representing a known disease or health status, thereby evaluating the treatment for said neurodegenerative disease.

In a preferred embodiment of the herein claimed methods, kits, recombinant animals, molecules, assays, and uses of the instant invention, said Myelin and lymphocyte proteolipid (MAL) gene and protein, is a member of the MAL family of proteins, is the Myelin and lymphocyte proteolipid (MAL) gene and protein MAL2. MAL2 is represented by the gene coding for the protein of the SwissProt Genbank accession number Q969L2. The amino acid sequence of said protein is deduced from the mRNA sequence corresponding to the cDNA sequence of Genbank accession number AY007723 and of the genomic DNA contig AC009514. In the instant invention MAL2 also refers to the nucleic acid sequence of SEQ ID NO: 2, coding for the protein of SEQ ID NO: 1 (Genbank accession number Q969L2) and to SEQ ID NO:4 which corresponds to the coding sequence of MAL2 (MAL2cds). In the instant invention said sequences are “isolated” as the term is employed herein. Further, in the instant invention, the gene coding for said MAL2 protein is also generally referred to as the MAL2 gene, or simply MaL2, and the protein of MAL2 is also generally referred to as the MAL2 protein, or simply MAL2.

In a further preferred embodiment of the herein claimed methods, kits, recombinant animals, molecules, assays, and uses of the instant invention, said neurodegenerative disease or disorder is Alzheimer's disease, and said subjects suffer from Alzheimer's disease.

It is preferred that the sample to be analyzed and determined is selected from the group comprising brain tissue or other tissues, or body cells. The sample can also comprise cerebrospinal fluid or other body fluids including saliva, urine, blood, serum plasma, or mucus. Preferably, the methods of diagnosis, prognosis, monitoring the progression or evaluating a treatment for a neurodegenerative disease, according to the instant invention, can be practiced ex corpore, and such methods preferably relate to samples, for instance, body fluids or cells, removed, collected, or isolated from a subject or patient or healthy control person.

In further preferred embodiments, said reference value is that of a level, or an activity, or both said level and said activity of (i) a transcription product of the gene coding for MAL2 protein, and/or of (ii) a translation product of the gene coding for MAL2 protein, and/or of (iii) a fragment, or derivative, or variant of said transcription or translation product in a sample obtained from a subject not suffering from said neurodegenerative disease (healthy control person, control sample, control) or in a sample obtained from a subject suffering from a neurodegenerative disease, in particular Alzheimer's disease (patient sample, patient).

In preferred embodiments, an alteration in the level and/or activity of a transcription product of the gene coding for MAL2 protein and/or of a translation product of the gene coding for MAL2 protein and/or of a fragment, or derivative, or variant thereof in a sample cell, or tissue, or body fluid obtained from said subject relative to a reference value representing a known health status (control sample) indicates a diagnosis, or prognosis, or increased risk of becoming diseased with a neurodegenerative disease, particularly AD.

In further preferred embodiments, an equal or similar level and/or activity of a transcription product of the gene coding for a MAL2 protein and/or of a translation product of the gene coding for a MAL2 protein and/or of a fragment, or derivative, or variant thereof in a sample cell, or tissue, or body fluid obtained from a subject relative to a reference value representing a known disease status of a neurodegenerative disease, in particular Alzheimer's disease (AD patient sample), indicates a diagnosis, or prognosis, or increased risk of becoming diseased with said neurodegenerative disease.

In preferred embodiments, measurement of the level of transcription products of the gene coding for MAL2 protein is performed in a sample obtained from a subject using a quantitative PCR-analysis with primer combinations to amplify said gene specific sequences from cDNA obtained by reverse transcription of RNA extracted from a sample of a subject. A Northern blot with probes specific for said gene can also be applied. It might further be preferred to measure transcription products by means of chip-based microarray technologies. These techniques are known to those of ordinary skill in the art (see Sambrook and Russell, Molecular Cloning: A Laboratory Manual, Cold Spring Harbor Laboratory Press, Cold Spring Harbor, N.Y., 2001; Schena M., Microarray Biochip Technology, Eaton Publishing, Natick, Mass., 2000). An example of an immunoassay is the detection and measurement of enzyme activity as disclosed and described in the patent application WO 02/14543.

Furthermore, a level and/or an activity of a translation product of the gene coding for MAL2 protein and/or of a fragment, or derivative, or variant of said translation product, and/or the level of activity of said translation product, and/or of a fragment, or derivative, or variant thereof, can be detected using an immunoassay, an activity assay, and/or a binding assay. These assays can measure the amount of binding between said protein molecule and an anti-protein antibody by the use of enzymatic, chromodynamic, radioactive, magnetic, or luminescent labels which are attached to either the anti-protein antibody or a secondary antibody which binds the anti-protein antibody. In addition, other high affinity ligands may be used. Immunoassays which can be used include e.g. ELISAs, Western blots and other techniques known to those of ordinary skill in the art (see Harlow and Lane, Antibodies: A Laboratory Manual, Cold Spring Harbor Laboratory Press, Cold Spring Harbor, N.Y., 1999 and Edwards R, Immunodiagnostics: A Practical Approach, Oxford University Press, Oxford; England, 1999). All these detection techniques may also be employed in the format of microarrays, protein-arrays, antibody microarrays, tissue microarrays, electronic biochip or protein-chip based technologies (see Schena M., Microarray Biochip Technology, Eaton Publishing, Natick, Mass., 2000).

In a preferred embodiment, the level, or the activity, or both said level and said activity of (i) a transcription product of the gene coding for MAL2 protein, and/or of (ii) a translation product of the gene coding MAL2 protein, and/or of (iii) a fragment, or derivative, or variant of said transcription or translation product in a series of samples taken from said subject over a period of time is compared, in order to monitor the progression of said disease. In further preferred embodiments, said subject receives a treatment prior to one or more of said sample gatherings. In yet another preferred embodiment, said level and/or activity is determined before and after said treatment of said subject.

In another aspect, the invention features a kit for diagnosing or prognosticating neurodegenerative diseases, in particular AD, in a subject, or determining the propensity or predisposition of a subject to develop a neurodegenerative disease, in particular AD, said kit comprising:

(a) at least one reagent which is selected from the group consisting of (i) reagents that selectively detect a transcription product of the gene coding for MAL2 protein (ii) reagents that selectively detect a translation product of the gene coding for MAL2 protein; and

(b) an instruction for diagnosing, or prognosticating a neurodegenerative disease, in particular AD, or determining the propensity or predisposition of a subject to develop such a disease by describing the steps of:

detecting a level, or an activity, or both said level and said activity, of said transcription product and/or said translation product of the gene coding for MAL2 protein, in a sample obtained from said subject; and
diagnosing or prognosticating a neurodegenerative disease, in particular AD, or determining the propensity or predisposition of said subject to develop such a disease, wherein a varied or altered level, or activity, or both said level and said activity, of said transcription product and/or said translation product of the gene coding for MAL2 protein compared to a reference value representing a known health status (control) and/or wherein a level, or activity, or both said level and said activity, of said transcription product and/or said translation product of the gene coding for MAL2 protein is similar or equal to a reference value representing a known disease status, preferably a disease status of AD, indicates a diagnosis or prognosis of a neurodegenerative disease, in particular AD, or an increased propensity or predisposition of developing such a disease. The kit, according to the present invention, may be particularly useful for the identification of individuals that are at risk of developing a neurodegenerative disease, in particular AD. Reagents that selectively detect a transcription product and/or a translation product of the gene coding for MAL2 protein are selected from the group of antibodies and/or primers and/or probes unique to the amino acid and/or nucleic acid sequences of MAL2 as described in the instant invention.

In a further aspect the invention features the use of a kit in a method of diagnosing or prognosticating a neurodegenerative disease, in particular Alzheimer's disease, in a subject, and in a method of determining the propensity or predisposition of a subject to develop such a disease, wherein the method comprises the steps of: (i) detecting in a sample obtained from said subject a level, or an activity, or both said level and said activity of a transcription product and/or of a translation product of a gene coding for MAL2, and (ii) comparing said level or activity, or both said level and said activity of a transcription product and/or of a translation product of a gene coding for MAL2 to a reference value representing a known health status and/or to a reference value representing a known disease status, and said level, or activity, or both said level and said activity, of said transcription product and/or said translation product is varied compared to a reference value representing a known health status, and/or is similar or equal to a reference value representing a known disease status.

Consequently, the kit, according to the present invention, may serve as a means for targeting identified individuals for early preventive measures or therapeutic intervention prior to disease onset, before irreversible damage in the course of the disease has been inflicted. Furthermore, in preferred embodiments, the kit featured in the invention is useful for monitoring a progression of a neurodegenerative disease, in particular AD in a subject, as well as monitoring success or failure of therapeutic treatment for such a disease of said subject.

In another aspect, the invention features a method of treating or preventing a neurodegenerative disease, in particular AD, in a subject comprising the administration to said subject in a therapeutically or prophylactically effective amount of an agent or agents which directly or indirectly affect a level, or an activity, or both said level and said activity, of (i) the gene coding for MAL2 protein, and/or (ii) a transcription product of the gene coding for MAL2 protein, and/or (iii) a translation product of the gene coding for MAL2 protein, and/or (iv) a fragment, or derivative, or variant of (i) to (iii). Said agent may comprise a small molecule, or it may also comprise a peptide, an oligopeptide, or a polypeptide. Said peptide, oligopeptide, or polypeptide may comprise an amino acid sequence of a translation product of the gene coding for MAL2 protein, or a fragment, or derivative, or a variant thereof. An agent for treating or preventing a neurodegenerative disease, in particular AD, according to the instant invention, may also consist of a nucleotide, an oligonucleotide, or a polynucleotide. Said oligonucleotide or polynucleotide may comprise a nucleotide sequence of the gene coding for MAL2 protein, either in sense orientation or in antisense orientation.

In preferred embodiments, the method comprises the application of per se known methods of gene therapy and/or antisense nucleic acid technology to administer said agent or agents. In general, gene therapy includes several approaches: molecular replacement of a mutated gene, addition of a new gene resulting in the synthesis of a therapeutic protein, and modulation of endogenous cellular gene expression by recombinant expression methods or by drugs. Gene-transfer techniques are described in detail (see e.g. Behr, Acc Chem Res 1993, 26: 274-278 and Mulligan, Science 1993, 260: 926-931) and include direct gene-transfer techniques such as mechanical microinjection of DNA into a cell as well as indirect techniques employing biological vectors (like recombinant viruses, especially retroviruses) or model liposomes, or techniques based on transfection with DNA coprecipitation with polycations, cell membrane pertubation by chemical (solvents, detergents, polymers, enzymes) or physical means (mechanic, osmotic, thermic, electric shocks). The postnatal gene transfer into the central nervous system has been described in detail (see e.g. Wolff, Curr Opin Neurobiol 1993, 3: 743-748).

In particular, the invention features a method of treating or preventing a neurodegenerative disease by means of antisense nucleic acid therapy, i.e. the down-regulation of an inappropriately expressed or defective gene by the introduction of antisense nucleic acids or derivatives thereof into certain critical cells (see e.g. Gillespie, DN&P 1992, 5: 389-395; Agrawal and Akhtar, Trends Biotechnol 1995, 13: 197-199; Crooke, Biotechnology 1992, 10: 882-6). Apart from hybridization strategies, the application of ribozymes, i.e. RNA molecules that act as enzymes, destroying RNA that carries the message of disease has also been described (see e.g. Barinaga, Science 1993, 262:1512-1514). In preferred embodiments, the subject to be treated is a human, and therapeutic antisense nucleic acids or derivatives thereof are directed against transcription products of the gene coding for MAL2 protein. It is preferred that cells of the central nervous system, preferably the brain, of a subject are treated in such a way. Cell penetration can be performed by known strategies such as coupling of antisense nucleic acids and derivatives thereof to carrier particles, or the above described techniques. Strategies for administering targeted therapeutic oligo-deoxynucleotides are known to those of skill in the art (see e.g. Wickstrom, Trends Biotechnol 1992, 10: 281-287). In some cases, delivery can be performed by mere topical application. Further approaches are directed to intracellular expression of antisense RNA. In this strategy, cells are transformed ex vivo with a recombinant gene that directs the synthesis of an RNA that is complementary to a region of target nucleic acid. Therapeutical use of intracellularly expressed antisense RNA is procedurally similar to gene therapy. A recently developed method of regulating the intracellular expression of genes by the use of double-stranded RNA, known variously as RNA interference (RNAi), can be another effective approach for nucleic acid therapy (Hannon, Nature 2002, 418: 244-251).

In further preferred embodiments, the method comprises grafting donor cells into the central nervous system, preferably the brain, of said subject, or donor cells preferably treated so as to minimize or reduce graft rejection, wherein said donor cells are genetically modified by insertion of at least one transgene encoding said agent or agents. Said transgene might be carried by a viral vector, in particular a retroviral vector. The transgene can be inserted into the donor cells by a nonviral physical transfection of DNA encoding a transgene, in particular by microinjection. Insertion of the transgene can also be performed by electroporation, chemically mediated transfection, in particular calcium phosphate transfection or liposomal mediated transfection (see McCelland and Pardee, Expression Genetics: Accelerated and High-Throughput Methods, Eaton Publishing, Natick, Mass., 1999).

In preferred embodiments, said agent for treating and preventing a neurodegenerative disease, in particular AD, is a therapeutic protein which can be administered to said subject, preferably a human, by a process comprising introducing subject cells into said subject, said subject cells having been treated in vitro to insert a DNA segment encoding said therapeutic protein, said subject cells expressing in vivo in said subject a therapeutically effective amount of said therapeutic protein. Said DNA segment can be inserted into said cells in vitro by a viral vector, in particular a retroviral vector.

Methods of treatment, according to the present invention, comprise the application of therapeutic cloning, transplantation, and stem cell therapy using embryonic stem cells or embryonic germ cells and neuronal adult stem cells, combined with any of the previously described cell- and gene therapeutic methods. Stem cells may be totipotent or pluripotent. They may also be organ-specific. Strategies for repairing diseased and/or damaged brain cells or tissue comprise (i) taking donor cells from an adult tissue. Nuclei of those cells are transplanted into unfertilized egg cells from which the genetic material has been removed. Embryonic stem cells are isolated from the blastocyst stage of the cells which underwent somatic cell nuclear transfer. Use of differentiation factors then leads to a directed development of the stem cells to specialized cell types, preferably neuronal cells (Lanza et al., Nature Medicine 1999, 9: 975-977), or (ii) purifying adult stem cells, isolated from the central nervous system, or from bone marrow (mesenchymal stem cells), for in vitro expansion and subsequent grafting and transplantation, or (iii) directly inducing endogenous neural stem cells to proliferate, migrate, and differentiate into functional neurons (Peterson DA, Curr. Opin. Pharmacol. 2002, 2:34-42). Adult neural stem cells are of great potential for repairing damaged or diseased brain tissues, as the germinal centers of the adult brain are free of neuronal damage or dysfunction (Colman A, Drug Discovery World 2001, 7: 66-71).

In preferred embodiments, the subject for treatment or prevention, according to the present invention, can be a human, an experimental animal, e.g. a mouse or a rat, a domestic animal, or a non-human primate. The experimental animal can be an animal model for a neurodegenerative disorder, e.g. a transgenic mouse and/or a knock-out mouse with an AD-type neuropathology.

In a further aspect, the invention features a modulator of an activity, or a level, or both said activity and said level of at least one substance which is selected from the group consisting of (i) the gene coding for MAL2 protein, and/or (ii) a transcription product of the gene coding for MAL2 protein, and/or (iii) a translation product of the gene coding for MAL2 protein, and/or (iv) a fragment, or derivative, or variant of (i) to (iii).

In an additional aspect, the invention features a pharmaceutical composition comprising said modulator and preferably a pharmaceutical carrier. Said carrier refers to a diluent, adjuvant, excipient, or vehicle with which the modulator is administered.

In a further aspect, the invention features a modulator of an activity, or a level, or both said activity and said level of at least one substance which is selected from the group consisting of (i) the gene coding for MAL2 protein, and/or (ii) a transcription product of the gene coding MAL2 protein, and/or (iii) a translation product of the gene coding for MAL2 protein, and/or (iv) a fragment, or derivative, or variant of (i) to (iii) for use in a pharmaceutical composition.

In another aspect, the invention provides for the use of a modulator of an activity, or a level, or both said activity and said level of at least one substance which is selected from the group consisting of (i) the gene coding for MAL2 protein, and/or (ii) a transcription product of the gene coding for MAL2 protein, and/or (iii) a translation product of the gene coding for MAL2 protein, and/or (iv) a fragment, or derivative, or variant of (i) to (iii) for a preparation of a medicament for treating or preventing a neurodegenerative disease, in particular AD.

In one aspect, the present invention also provides a kit comprising one or more containers filled with a therapeutically or prophylactically effective amount of said pharmaceutical composition.

In a further aspect, the invention features a recombinant, non-human animal comprising a non-native MAL2 gene sequence, or a fragment, or a derivative, or variant thereof. The generation of said recombinant, non-human animal comprises (i) providing a gene targeting construct containing said gene sequence and a selectable marker sequence, and (ii) introducing said targeting construct into a stem cell of a non-human animal, and (iii) introducing said non-human animal stem cell into a non-human embryo, and (iv) transplanting said embryo into a pseudopregnant non-human animal, and (v) allowing said embryo to develop to term, and (vi) identifying a genetically altered non-human animal whose genome comprises a modification of said gene sequence in both alleles, and (vii) breeding the genetically altered non-human animal of step (vi) to obtain a genetically altered non-human animal whose genome comprises a modification of said endogenous gene, wherein said gene is mis-expressed, or under-expressed, or over-expressed, and wherein said disruption or alteration results in said non-human animal exhibiting a predisposition to developing symptoms of a neurodegenerative disease, in particular AD. Strategies and techniques for the generation and construction of such an animal are known to those of ordinary skill in the art (see e.g. Capecchi, Science 1989, 244: 1288-1292 and Hogan et al., Manipulating the Mouse Embryo: A Laboratory Manual, Cold Spring Harbor Laboratory Press, Cold Spring Harbor, N.Y., 1994 and Jackson and Abbott, Mouse Genetics and Transgenics: A Practical Approach, Oxford University Press, Oxford, England, 1999). It is preferred to make use of such a recombinant non-human animal as an animal model for investigating neurodegenerative diseases, in particular Alzheimer's disease. Such an animal may be useful for screening, testing and validating compounds, agents and modulators in the development of diagnostics and therapeutics to treat neurodegenerative diseases, in particular Alzheimer's disease.

In another aspect, the invention features an assay for screening for a modulator of neurodegenerative diseases, in particular AD, or related diseases and disorders of one or more substances selected from the group consisting of (i) the gene coding for MAL2 protein, and/or (ii) a transcription product of the gene coding for MAL2 protein, and/or (iii) a translation product of the gene coding for MAL2 protein, and/or (iv) a fragment, or derivative, or variant of (i) to (iii). This screening method comprises (a) contacting a cell with a test compound, and (b) measuring the activity, or the level, or both the activity and the level of one or more substances recited in (i) to (iv), and (c) measuring the activity, or the level, or both the activity and the level of said substances in a control cell not contacted with said test compound, and (d) comparing the levels of the substance in the cells of step (b) and (c), wherein an alteration in the activity and/or level of said substances in the contacted cells indicates that the test compound is a modulator of said diseases and disorders.

In one further aspect, the invention features a screening assay for a modulator of neurodegenerative diseases, in particular AD, or related diseases and disorders of one or more substances selected from the group consisting of (i) the gene coding for MAL2 protein, and/or (ii) a transcription product of the gene coding for MAL2 protein, and/or (iii) a translation product of the gene coding for MAL2 protein, and/or (iv) a fragment, or derivative, or variant of (i) to (iii), comprising (a) administering a test compound to a test animal which is predisposed to developing or has already developed symptoms of a neurodegenerative disease or related diseases or disorders, and (b) measuring the activity and/or level of one or more substances recited in (i) to (iv), and (c) measuring the activity and/or level of said substances in a matched control animal which is equally predisposed to developing or has already developed said symptoms of a neurodegenerative disease, and to which animal no such test compound has been administered, and (d) comparing the activity and/or level of the substance in the animals of step (b) and (c), wherein an alteration in the activity and/or level of substances in the test animal indicates that the test compound is a modulator of said diseases and disorders.

In a preferred embodiment, said test animal and/or said control animal is a recombinant, non-human animal which expresses the gene coding for MAL2 protein, or a fragment thereof, or a derivative thereof, under the control of a transcriptional regulatory element which is not the native MAL2 protein gene transcriptional control regulatory element.

In another embodiment, the present invention provides a method for producing a medicament comprising the steps of (i) identifying a modulator of neurodegenerative diseases by a method of the aforementioned screening assays and (ii) admixing the modulator with a pharmaceutical carrier. However, said modulator may also be identifiable by other types of screening assays.

In another aspect, the present invention provides for an assay for testing a compound, preferably for screening a plurality of compounds, for inhibition of binding between a ligand and MAL2 protein, or a fragment, or derivative, or variant thereof. Said screening assay comprises the steps of (i) adding a liquid suspension of said MAL2 protein, or a fragment, or derivative, or variant thereof, to a plurality of containers, and (ii) adding a compound or a plurality of compounds to be screened for said inhibition to said plurality of containers, and (iii) adding a detectable, preferably a fluorescently labelled ligand to said containers, and (iv) incubating said MAL2 protein, or said fragment, or derivative or variant thereof, and said compound or plurality of compounds, and said detectable, preferably fluorescently labelled ligand, and (v) measuring the amounts of preferably the fluorescence associated with said MAL2 protein, or with said fragment, or derivative, or variant thereof, and (vi) determining the degree of inhibition by one or more of said compounds of binding of said ligand to said MAL2 protein, or said fragment, or derivative, or variant thereof. It might be preferred to reconstitute said MAL2 translation product, or fragment, or derivative, or variant thereof into artificial liposomes to generate the corresponding proteoliposomes to determine the inhibition of binding between a ligand and said MAL2 translation product. Methods of reconstitution of MAL2 translation products from detergent into liposomes have been detailed (Schwarz et al., Biochemistry 1999, 38: 9456-9464; Krivosheev and Usanov, Biochemistry-Moscow 1997, 62: 1064-1073). Instead of utilizing a fluorescently labelled ligand, it might in some aspects be preferred to use any other detectable label known to the person skilled in the art, e.g. radioactive labels, and detect it accordingly. Said method may be useful for the identification of novel compounds as well as for evaluating compounds which have been improved or otherwise optimized in their ability to inhibit the binding of a ligand to a gene product of the gene coding for MAL2 protein, or a fragment, or derivative, or variant thereof. One example of a fluorescent binding assay, in this case based on the use of carrier particles, is disclosed and described in patent application WO 00/52451. A further example is the competitive assay method as described in patent WO 02/01226. Preferred signal detection methods for screening assays of the instant invention are described in the following patent applications: WO 96/13744, WO 98/16814, WO 98/23942, WO 99/17086, WO 99/34195, WO 00/66985, WO 01/59436 and WO 01/59416.

In one further embodiment, the present invention provides a method for producing a medicament comprising the steps of (i) identifying a compound as an inhibitor of binding between a ligand and a gene product of the gene coding for MAL2 protein by the aforementioned inhibitory binding assay and (ii) admixing the compound with a pharmaceutical carrier. However, said compound may also be identifiable by other types of screening assays.

In another aspect, the invention features an assay for testing a compound, preferably for screening a plurality of compounds to determine the degree of binding of said compounds to MAL2 protein, or to a fragment, or derivative, or variant thereof. Said screening assay comprises (i) adding a liquid suspension of said MAL2 protein, or a fragment, or derivative, or variant thereof, to a plurality of containers, and (ii) adding a detectable, preferably a fluorescently labelled compound or a plurality of detectable, preferably fluorescently labelled compounds to be screened for said binding to said plurality of containers, and (iii) incubating said MAL2 protein, or said fragment, or derivative, or variant thereof, and said detectable, preferably fluorescently labelled compound or detectable, preferably fluorescently labelled compounds, and (iv) measuring the amounts of preferably the fluorescence associated with said MAL2 protein, or with said fragment, or derivative, or variant thereof, and (v) determining the degree of binding by one or more of said compounds to said MAL2 protein, or said fragment, or derivative, or variant thereof. In this type of assay it might be preferred to use a fluorescent label. However, any other type of detectable label might also be employed. Also in this type of assay it might be preferred to reconstitute an MAL2 translation product or a fragment, or derivative, or variant thereof into artificial liposomes as described in the present invention. Said assay methods may be useful for the identification of novel compounds as well as for evaluating compounds which have been improved or otherwise optimized in their ability to bind to MAL2 protein, or a fragment, or derivative, or variant thereof.

In one further embodiment, the present invention provides a method for producing a medicament comprising the steps of (i) identifying a compound as a binder to a gene product of the gene coding for MAL2 protein by the aforementioned binding assays and (ii) admixing the compound with a pharmaceutical carrier. However, said compound may also be identifiable by other types of screening assays.

In another embodiment, the present invention provides for a medicament obtainable by any of the methods according to the herein claimed screening assays. In one further embodiment, the instant invention provides for a medicament obtained by any of the methods according to the herein claimed screening assays.

The present invention features a protein molecule and the use of said protein molecule as shown in SEQ ID NO:1, said protein molecule being a translation product of the gene coding for MAL2, or a fragment, or derivative, or variant thereof, as a diagnostic target for detecting a neurodegenerative disease, in particular Alzheimer's disease.

The present invention further features a protein molecule and the use of said protein molecule as shown in SEQ ID NO:1, said protein molecule being a translation product of the gene coding for MAL2, or a fragment, or derivative, or variant thereof, as a screening target for reagents or compounds preventing, or treating, or ameliorating a neurodegenerative disease, in particular Alzheimer's disease.

The present invention features an antibody which is specifically immunoreactive with an immunogen, wherein said immunogen is a translation product of the gene coding for MAL2 protein, SEQ ID NO:1, or a fragment, or derivative, or variant thereof. The immunogen may comprise immunogenic or antigenic epitopes or portions of a translation product of said gene, wherein said immunogenic or antigenic portion of a translation product is a polypeptide, and wherein said polypeptide elicits an antibody response in an animal, and wherein said polypeptide is immunospecifically bound by said antibody. Methods for generating antibodies are well known in the art (see Harlow et al., Antibodies, A Laboratory Manual, Cold Spring Harbor Laboratory Press, Cold Spring Harbor, N.Y., 1988). The term “antibody”, as employed in the present invention, encompasses all forms of antibodies known in the art, such as polyclonal, monoclonal, chimeric, recombinatorial, anti-idiotypic, humanized, or single chain antibodies, as well as fragments thereof (see Dubel and Breitling, Recombinant Antibodies, Wiley-Liss, New York, N.Y., 1999). Antibodies of the present invention are useful, for instance, in a variety of diagnostic and therapeutic methods, based on state-in-the-art techniques (see Harlow and Lane, Using Antibodies: A Laboratory Manual, Cold Spring Harbor Laboratory Press, Cold Spring Harbor, N.Y., 1999 and Edwards R., Immunodiagnostics: A Practical Approach, Oxford University Press, Oxford, England, 1999) such as enzyme-immuno assays (e.g. enzyme-linked immunosorbent assay, ELISA), radioimmuno assays, chemoluminescence-immuno assays, Western-blot, immunoprecipitation and antibody microarrays. These methods involve the detection of translation products of the MAL2 gene, or fragments, or derivatives, or variants thereof.

In a preferred embodiment of the present invention, said antibodies can be used for detecting the pathological state of a cell in a sample obtained from a subject, comprising immunocytochemical staining of said cell with said antibody, wherein an altered degree of staining, or an altered staining pattern in said cell compared to a cell representing a known health status indicates a pathological state of said cell. Preferably, the pathological state relates to a neurodegenerative disease, in particular to AD. Immunocytochemical staining of a cell can be carried out by a number of different experimental methods well known in the art. It might be preferred, however, to apply an automated method for the detection of antibody binding, wherein the determination of the degree of staining of a cell, or the determination of the cellular or subcellular staining pattern of a cell, or the topological distribution of an antigen on the cell surface or among organelles and other subcellular structures within the cell, are carried out according to the method described in US patent 6150173.

Other features and advantages of the invention will be apparent from the following description of figures and examples which are illustrative only and not intended to limit the remainder of the disclosure in any way.

### FIGURES

FIG. 1 discloses the initial identification of the differential expression of the gene coding for MAL2 protein in a fluorescence differential display screen. The figure shows a clipping of a large preparative fluorescent differential display gel. PCR products from the frontal cortex (F) and the temporal cortex (T) of two healthy control subjects and six AD patients were loaded in duplicate onto a denaturing polyacrylamide gel (from left to right). PCR products were obtained by amplification of the individual cDNAs with the corresponding one-base-anchor oligonucleotide and the specific Cy3 labelled random primers. The arrow indicates the migration position where significant differences in intensity of the signals for a transcription product of the gene coding for MAL2 protein derived from frontal cortex and from the temporal cortex of AD patients as compared to healthy controls exist. The differential expression reflects a down-regulation of MAL2 gene transcription in the temporal cortices of AD patients compared to the temporal cortices of control persons.

FIG. 2 and FIG. 3 illustrate the verification of the differential expression of the MAL2 gene in AD brain tissues by quantitative RT-PCR analysis. Quantification of RT-PCR products from RNA samples collected from the frontal cortex (F) and the temporal cortex (T) of AD patients (FIG. 2A) and samples from the frontal cortex (F) and the hippocampus (H) of AD patients (FIG. 3A) was performed by the LightCycler rapid thermal cycling technique. Likewise, samples of healthy, age-matched control individuals were compared (FIG. 2B for frontal cortex and temporal cortex, FIG. 3B for frontal cortex and hippocampus). The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figures depict the kinetics of amplification by plotting the cycle number against the amount of amplified material as measured by its fluorescence. Note that the amplification kinetics of MAL2 cDNAs from both, the frontal and temporal cortices of a normal control individual, and from the frontal cortex and hippocampus of a normal control individual, respectively, during the exponential phase of the reaction are juxtaposed (FIGS. 2B and 3B, arrowhead), whereas in Alzheimer's disease (FIGS. 2A and 3A, arrowhead) there is a significant separation of the corresponding curves, indicating a differential expression of the gene coding for MAL2 in the respective analyzed brain regions. The differential expression reflects a dysregulation, preferably a down-regulation of a transcription product of the human MAL2 gene, or a fragment, or derivative, or variant thereof, in the temporal cortex relative to the frontal cortex.

FIG. 4 illustrates the verification of the differential expression of the human MAL2 gene in AD brain tissues (P) versus healthy control brain tissue samples (C) by quantitative RT-PCR analysis. Quantification of RT-PCR products from RNA samples collected from the frontal cortex region of AD patients and of healthy, age-matched control persons (P_{(F)}−C_{(F)}; FIG. 5A) was performed by the LightCycler rapid thermal cycling technique. Likewise, samples from the temporal cortex region of AD patients and of control individuals (P_{(T)}−C_{(T)}; FIG. 5B) were compared. The data were normalized to the combined average values of a set of standard genes which showed no significant differences in their gene expression levels. Said set of standard genes consisted of genes for cyclophilin B, the ribosomal protein S9, the transferrin receptor, GAPDH, and beta-actin. The figures depict the kinetics of amplification by plotting the cycle number against the amount of amplified material as measured by its fluorescence. The curves delineating the amplification kinetics of MAL2 cDNAs are significantly separated during the exponential phase of the amplification reaction, for both brain regions analyzed: (i) frontal cortex of a normal control individual in comparison to frontal cortex of an AD patient (FIG. 5A), and (ii) temporal cortex of a normal control individual in comparison to temporal cortex of an AD patient (FIG. 5B). This indicates a differential expression of the gene coding for MAL2 in the analyzed brain regions of AD patients in comparison with healthy control persons and reflects a down-regulation of a transcription product of the human MAL2 gene, or a fragment, or derivative, or variant thereof, in the temporal cortex and in the frontal cortex of AD patients relative to the temporal cortex and the frontal cortex of healthy control persons.

FIG. 5 discloses SEQ ID NO: 1, the amino acid sequence of the human MAL2 protein. The full length human MAL2 protein comprises 176 amino acids (aa), as defined by the SwissProt accession number Q969L2.

FIG. 6 shows SEQ ID NO: 2, the nucleotide sequence of the human MAL2 cDNA, comprising 2808 nucleotides (nt), as defined by the Genbank accession number AY007723.

FIG. 7 depicts SEQ ID NO: 3, the nucleotide sequence of the 270 bp MAL2 cDNA fragment, identified and obtained by differential display and subsequent cloning (sequence in 5′ to 3′ direction).

FIG. 8 shows the nucleotide sequence of SEQ ID NO: 4, the coding sequence (cds) of the human MAL2 gene, comprising 531 nucleotides (nucleotides 80-610 of SEQ ID NO: 2).

FIG. 9 outlines the sequence alignment of SEQ ID NO: 3 to the nucleotide sequence of MAL2 cDNA (SEQ ID NO: 2).

FIG. 10 lists MAL2 gene expression levels in the temporal cortex relative to the frontal cortex in fifteen AD patients, herein identified by internal reference numbers P010, P011, P012, P014, P016, P017, P019, P038, P040, P041, P042, P046, P047, P048, P049 (1.02 to 3.45 fold, values according to the formula described below) and twentyfive healthy, age-matched control individuals, herein identified by internal reference numbers C005, C008, C011, C012, C014, C025, C026, C027, C028, C029, C030, C031, C032, C033, C034, C035, C036, C038, C039, C041, C042, DE02, DE03, DE05, DE07 (0.29 to 3.70 fold, values according to the formula described below). For an up-regulation in the temporal cortex, the values shown are calculated according to the formula described herein (see below) and in case of an up-regulation in the frontal cortex the reciprocal values of the formula described herein are calculated, respectively. The bar diagram visualizes individual natural logarithmic values of the temporal to frontal cortex, In(IT/IF), and of the frontal to temporal cortex regulation factors, In(IF/IT), in different Braak stages (0 to 6). An obvious difference reflecting a down-regulation in the temporal cortex is shown. The Braak stages correlate with the progressive course of AD disease which, as shown in the instant invention, is associated with an increasing difference in the regulation, the level and the activity of MAL2 as described above.

FIG. 11 lists the gene expression levels in the hippocampus relative to the frontal cortex for the MAL2 gene in six Alzheimer's disease patients, herein identified by internal reference numbers P010, P011, P012, P014, P016, P019 (0.12 to 2.04 fold) and three healthy, age-matched control individuals, herein identified by internal reference numbers C004, C005, C008 (0.62 to 1.00 fold). The values shown are calculated according to the formula described herein (see below). The scatter diagram visualizes individual logarithmic values of the hippocampus to frontal cortex regulation ratios, log(ratio HC/IF), in control samples (dots) and in AD patient samples (triangles).

FIG. 12 shows the analysis of absolute mRNA expression of MAL2 (alias ens0711) by comparison of control and AD stages using statistical method of the median at 98%-confidence level. The data were calculated by defining control groups including subjects with either Braak stages 0 to 1, Braak stages 0 to 2, or Braak stages 0 to 3 which are compared with the data calculated for the defined AD patient groups including Braak stages 2 to 6, Braak stages 3 to 6 and Braak stages 4 to 6, respectively. Additionally, three groups including subjects with either Braak stages 0 to 1, Braak stages 2 to 3 and Braak stages 4 to 6, respectively, were compared with each other. A difference was detected comparing frontal cortex (F) and inferior temporal cortex (T) of AD patients and of healthy age-matched control persons with each other. Said difference reflects a down-regulation of MAL2 in the temporal cortex and in the frontal cortex of AD patients relative to the temporal cortex and frontal cortex of healthy age-matched control persons.

FIG. 13 depicts a Western blot image of total cell protein extracts labeled with polyclonal anti-myc antibody (MBL, 1:1000).

Lanes A and B: total protein extract of H4APPsw cells stably expressing MAL2 tagged with a myc-tag (MAL2-myc, A) and control H4APPsw cells (B). The arrow indicates a major band at about 19 kDa (lane A), which corresponds to the predicted molecular weight of the MAL2 protein.

FIG. 14 shows the immunofluorescence analysis of H4APPsw control cells and H4APPsw cells stably over-expressing the myc-tagged MAL protein (H4APPsw-MAL2 cds-myc). The MAL2-myc protein was detected with rabbit polyclonal anti-myc antibodies (MBL) and a Cy3-conjugated anti-rabbit antibody (Amersham) (FIGS. 14A and 14B). The cellular nucleus was stained with DAPI (FIGS. 14C and 14D). The overlay analysis indicate that the MAL2-myc protein is mainly localized to the golgi and the plasma membrane (FIG. 14E) and is over-expressed in more than 70% of the H4APPsw-MAL2cds-myc transduced cells as compared to the H4APPsw control cells (FIG. 14F).

FIG. 15 depicts sections from human frontal cortex labeled with an affinity-purified rabbit polyclonal anti-MAL2 antibodies (green signals) raised against a peptide corresponding to amino acids 22-38 of MAL2. Immunoreactivity of MAL2 was observed in both the cerebral cortex (CT) and the white matter (WM) (FIG. 15A, low magnification). MAL2 immunoreactivity was observed mainly in the cytoplasm and also in plasma membranes of neurons and glial cells in the cortex (FIG. 15B, high magnification). FIG. 15C shows MAL2 colocalization in the white matter with CNPase (2′,3′-cyclic nucleotide 3′-phosphodiesterase, red signals) in oligodendrocytic cell bodies (indicated by arrows) and to a lesser extent in myelin sheets. Blue signals indicate nuclei stained with DAPI.

### EXAMPLE I

(i) Brain Tissue Dissection from Patients with Ad:

Brain tissues from AD patients and healthy, age-matched control subjects were collected, on average, within 6 hours post-mortem and immediately frozen on dry ice. Sample sections from each tissue were fixed in paraformaldehyde for histopathological confirmation of the diagnosis. Brain areas for differential expression analysis were identified and stored at −80° C. until RNA extractions were performed. Tissues of several brain regions, the frontal cortex (F), the temporal cortex (T) and the hippocampus (H), which exhibit selective vulnerability to neuronal loss and degeneration in AD, were used for the herein disclosed examples.

(ii) Isolation of Total mRNA:

Total RNA was extracted from post-mortem brain tissue by using the RNeasy kit (Qiagen) according to the manufacturer's protocol. The accurate RNA concentration and the RNA quality were determined with the DNA LabChip system using the Agilent 2100 Bioanalyzer (Agilent Technologies). For additional quality testing of the prepared RNA, i.e. exclusion of partial degradation and testing for DNA contamination, specifically designed intronic GAPDH oligonucleotides and genomic DNA as reference control were utilised to generate a melting curve with the LightCycler technology as described in the supplied protocol by the manufacturer (Roche).

(iii) cDNA Synthesis and Identification of Differentially Expressed Genes by Fluorescence differential display (FDD):

In order to identify changes in gene expression in different tissue, a modified and improved differential display (DD) screening method was employed. The original DD screening method is known to those skilled in the art (Liang and Pardee, Science 1995, 267:1186-7). This technique compares two populations of RNA and provides clones of genes that are expressed in one population but not in the other. Several samples can be analyzed simultaneously and both up- and down-regulated genes can be identified in the same experiment. By adjusting and refining several steps in the DD method as well as modifying technical parameters, e.g. increasing redundancy, evaluating optimized reagents and conditions for reverse transcription of total RNA, optimizing polymerase chain reactions (PCR) and separation of the products thereof, a technique was developed which allows for highly reproducible and sensitive results. The applied and improved DD technique was described in detail by von der Kammer et al. (Nucleic Acids Research 1999, 27: 2211-2218). A set of 64 specifically designed random primers were developed (standard set) to achieve a statistically comprehensive analysis of all possible RNA species. Further, the method was modified to generate a preparative DD slab-gel technique, based on the use of fluorescently labelled primers. In the present invention, RNA populations from carefully selected post-mortem brain tissues (frontal and temporal cortex) of Alzheimer's disease patients and age-matched control subjects were compared.

As starting material for the DD analysis we used total RNA, extracted as described above (ii). Equal amounts of 0.05 μg RNA each were transcribed into cDNA in 20 μl reactions containing 0.5 mM each dNTP, 1 μl Sensiscript Reverse Transcriptase and 1× RT buffer (Qiagen), 10 U RNase inhibitor (Qiagen) and 1 μM of either one-base-anchor oligonucleotides HT₁₁A, HT₁₁G or HT₁₁C (Liang et al., Nucleic Acids Research 1994, 22: 5763-5764; Zhao et al., Biotechniques 1995, 18: 842-850). Reverse transcription was performed for 60 min at 37° C. with a final denaturation step at 93° C. for 5 min. 2 μl of the obtained cDNA each was subjected to a polymerase chain reaction (PCR) employing the corresponding one-base-anchor oligonucleotide (1 μM) along with either one of the Cy3 labelled random DD primers (1 μM), 1× GeneAmp PCR buffer (Applied Biosystems), 1.5 mM MgCl₂ (Applied Biosystems), 2 μM dNTP-Mix (dATP, dGTP, dCTP, dTTP Amersham Pharmacia Biotech), 5% DMSO (Sigma), 1 U AmpliTaq DNA Polymerase (Applied Biosystems) in a 20 μl final volume. PCR conditions were set as follows: one round at 94° C. for 30 sec for denaturing, cooling 1° C./sec down to 40° C., 40° C. for 4 min for low-stringency annealing of primer, heating 1° C./sec up to 72° C., 72° C. for 1 min for extension. This round was followed by 39 high-stringency cycles: 94° C. for 30 sec, cooling 1° C./sec down to 60° C., 60° C. for 2 min, heating 1° C./sec up to 72° C., 72° C. for 1 min. One final step at 72° C. for 5 min was added to the last cycle (PCR cycler: Multi Cycler PTC 200, MJ Research). 8 μl DNA loading buffer were added to the 20 μl PCR product preparation, denatured for 5 min and kept on ice until loading onto a gel. 3.5 μl each were separated on 0.4 mm thick, 6% polyacrylamide (Long Ranger)/7 M urea sequencing gels in a slab-gel system (Hitachi Genetic Systems) at 2000 V, 60 W, 30 mA, for 1 h 40 min. Following completion of the electrophoresis, gels were scanned with a FMBIO II fluorescence-scanner (Hitachi Genetic Systems), using the appropriate FMBIO II Analysis 8.0 software. A full-scale picture was printed, differentially expressed bands marked, excised from the gel, transferred into 1.5 ml containers, overlayed with 200 μl sterile water and kept at −20° C. until extraction.

Elution and reamplification of DD products: The differential bands were extracted from the gel by boiling in 200 μl H₂O for 10 min, cooling down on ice and precipitation from the supernatant fluids by using ethanol (Merck) and glycogen/sodium acetate (Merck) at −20° C. over night, and subsequent centrifugation at 13.000 rpm for 25 min at 4° C. Pellets were washed twice in ice-cold ethanol (80%), resuspended in 10 mM Tris pH 8.3 (Merck) and dialysed against 10% glycerol (Merck) for 1 h at room temperature on a 0.025 μm VSWP membrane (Millipore). The obtained preparations were used as templates for reamplification by 15 high-stringency cycles in 25-μl PCR mixtures containing the corresponding primer pairs as used for the DD PCR (see above) under identical conditions, with the exception of the initial round at 94° C. for 5 min, followed by 15 cycles of: 94° C. for 45 sec, 60° C. for 45 sec, ramp 1° C./sec to 70° C. for 45 sec, and one final step at 72° C. for 5 min.

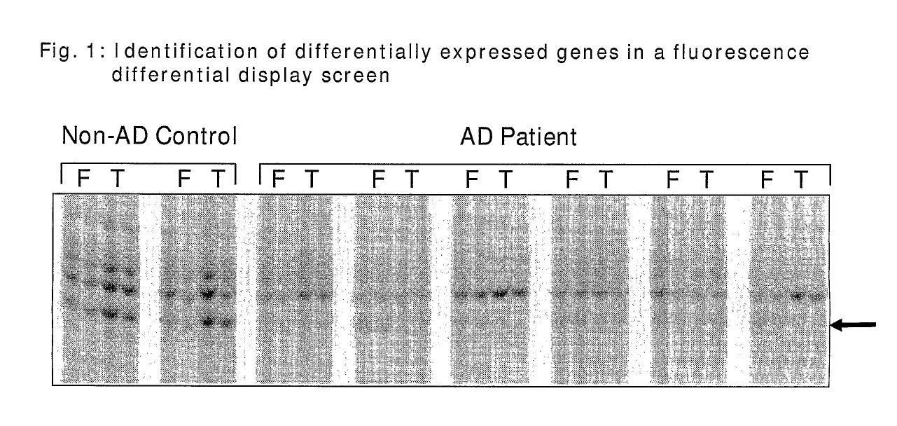

Cloning and sequencing of DD products: Re-amplified cDNAs were analyzed with the DNA LabChip system (Agilent 2100 Bioanalyzer, Agilent Technologies) and ligated into the pCR-Blunt II-TOPO vector and transformed into E. coli Top10F′ cells (Zero Blunt TOPO PCR Cloning Kit, Invitrogen) according to the manufacturer's instructions. Cloned cDNA fragments were sequenced by commercially available sequencing facilities. The result of one such FDD experiment for the gene coding for MAL2 protein is shown in FIG. 1.

(iv) Confirmation of differential expression by quantitative RT-PCR:

Positive corroboration of differential MAL2 gene expression was performed using the LightCycler technology (Roche). This technique features rapid thermal cyling for the polymerase chain reaction as well as real-time measurement of fluorescent signals during amplification and therefore allows for highly accurate quantification of RT-PCR products by using a kinetic, rather than endpoint readout. The ratios of MAL2 cDNAs from temporal cortices of AD patients and of healthy age-matched control individuals, from the frontal cortices of AD patients and of healthy age-matched control individuals, from the hippocampi of AD patients and of age-matched control individuals, and the ratios of MAL2 cDNAs from the temporal cortex and frontal cortex of AD patients and of healthy age-matched control individuals, and the ratios of MAL2 cDNAs from the hippocampus and from frontal cortex of AD patients and of healthy age-matched control individuals, respectively, were determined (relative quantification). The mRNA expression profiling between frontal cortex tissue (F) and inferior temporal cortex tissue (T) of MAL2 has been analyzed in four up to nine tissues per Braak stage. Because of the lack of high quality tissues from one donor with Braak 3 pathology, tissues of one additional donor with Braak 2 pathology were included, and because of the lack of high quality tissues from one donor with Braak 6 pathology, tissue samples of one additional donor with Braak 5 pathology were included.

For the analysis of the profiling, two general approaches have been applied. Both comparative profiling studies, frontal cortex against inferior temporal cortex as well as control against AD patients, which contribute to the complex view of the relevance of MAL2 in AD physiology, are shown in detail below.

1) Relative Comparison of the mRNA Expression Between Frontal Cortex Tissue and Inferior Temporal Cortex Tissue of Controls and of AD Patients.

This approach allowed to verify that MAL2 is either involved in the protection of the less vulnerable tissue (frontal cortex) against degeneration, or is involved in or enhances the process of degeneration in the more vulnerable tissue (inferior temporal cortex).

First, a standard curve was generated to determine the efficiency of the PCR with specific primers for the gene coding for MAL2:

SEQ ID NO:5: 5′-ACCTGTAGAGATCCTCGTCATGG-3′
(nucleotides 1930-1952 of SEQ ID NO:2)
and
SEQ ID NO:6: 5′-TGGCCTCACTCTTACTTGTCCTT-3′
(complementary nucleotides 2000-1978 of
SEQ ID NO:2).

PCR amplification (95° C. and 1 sec, 56° C. and 5 sec, and 72° C. and 5 sec) was performed in a volume of 20 μl containing LightCycler-FastStart DNA Master SYBR Green I mix (contains FastStart Taq DNA polymerase, reaction buffer, dNTP mix with dUTP instead of dTTP, SYBR Green I dye, and 1 mM MgCl₂; Roche), 0.5 μM primers, 2 μl of a cDNA dilution series (final concentration of 40, 20, 10, 5, 1 and 0.5 ng human total brain cDNA; Clontech) and, depending on the primers used, additional 3 mM MgCl₂. Melting curve analysis revealed a single peak at approximately 81.5° C. with no visible primer dimers. Quality and size of the PCR product were determined with the DNA LabChip system (Agilent 2100 Bioanalyzer, Agilent Technologies). A single peak at the expected size of 71 bp for the gene coding for MAL2 protein was observed in the electropherogram of the sample.

In an analogous manner, the PCR protocol was applied to determine the PCR efficiency of a set of reference genes which were selected as a reference standard for quantification. In the present invention, the mean value of five such reference genes was determined: (1) cyclophilin B, using the specific primers SEQ ID NO:7: 5′-ACTGAAGCACTACGGGCCTG-3′ and SEQ ID NO:8: 5′-AGCCGTTGGTGTCTTTGCC-3′ except for MgCl₂ (an additional 1 mM was added instead of 3 mM). Melting curve analysis revealed a single peak at approximately 87° C. with no visible primer dimers. Agarose gel analysis of the PCR product showed one single band of the expected size (62 bp). (2) Ribosomal protein S9 (RPS9), using the specific primers SEQ ID NO:9: 5′-GGTCAAATTTACCCTGGCCA-3′ and SEQ ID NO:10: 5′-TCTCATCAAGCGTCAGCAGTTC-3′ (exception: additional 1 mM MgCl₂ was added instead of 3 mM). Melting curve analysis revealed a single peak at approximately 85° C. with no visible primer dimers. Agarose gel analysis of the PCR product showed one single band with the expected size (62 bp). (3) beta-actin, using the specific primers SEQ ID NO:11: 5′-TGGAACGGTGAAGGTGACA-3′ and SEQ ID NO:12: 5′-GGCAAGGGACTTCCTGTAA-3′. Melting curve analysis revealed a single peak at approximately 87° C. with no visible primer dimers. Agarose gel analysis of the PCR product showed one single band with the expected size (142 bp). (4) GAPDH, using the specific primers SEQ ID NO:13: 5′-CGTCATGGGTGTGAACCATG-3′ and SEQ ID NO:14: 5′-GCTAAGCAGTTGGTGGTGCAG-3′. Melting curve analysis revealed a single peak at approximately 83° C. with no visible primer dimers. Agarose gel analysis of the PCR product showed one single band with the expected size (81 bp). (5) Transferrin receptor TRR, using the specific primers SEQ ID NO:15: 5′-GTCGCTGGTCAGTTCGTGATT-3′ and SEQ ID NO:16: 5′-AGCAGTTGGCTGTTGTACCTCTC-3′. Melting curve analysis revealed a single peak at approximately 83° C. with no visible primer dimers. Agarose gel analysis of the PCR product showed one single band with the expected size (80 bp).

For calculation of the values, first the logarithm of the cDNA concentration was plotted against the threshold cycle number Cₜ for the gene coding for MAL2 protein and the five reference standard genes. The slopes and the intercepts of the standard curves (i.e. linear regressions) were calculated for all genes. In a second step, cDNAs from frontal cortex, temporal cortex and hippocampus, and cDNAs from frontal cortices of AD patients and of healthy control individuals, and from temporal cortices of AD patients and of healthy control individuals, respectively, were analyzed in parallel and normalized to cyclophilin B. The Cₜ values were measured and converted to ng total brain cDNA using the corresponding standard curves:
10 ˆ((Cₜ value−intercept)/slope) [ng total brain cDNA]

The values for temporal and frontal cortex and the values for hippocampus and frontal cortex MAL2 cDNAs, and the values from the frontal cortex MAL2 cDNAs of AD patients (P) and control individuals (C), and the values for temporal cortex MAL2 cDNAs of AD patients (P) and of healthy control individuals (C), respectively, were normalized to cyclophilin B and the ratios were calculated according to formulas:

$$Ratio = \frac{MAL 2 temporal [ ng ] / cyclophilin B temporal [ ng ]}{MAL 2 frontal [ ng ] / cyclophilin B frontal [ ng ]}$$

$$Ratio = \frac{\begin{matrix}MAL 2 hippocampus [ ng ] / \\ cyclophilin B hippocampus [ ng ]\end{matrix}}{MAL 2 frontal [ ng ] / cyclophilin B frontal [ ng ]}$$

$$Ratio = \frac{\begin{matrix}MAL 2 ( P ) temporal [ ng ] / \\ cyclophilin B ( P ) temporal [ ng ]\end{matrix}}{\begin{matrix}MAL 2 ( C ) temporal [ ng ] / \\ cyclophilin B ( C ) temporal [ ng ]\end{matrix}}$$

$$Ratio = \frac{MAL 2 ( P ) frontal [ ng ] / cyclophilin B ( P ) frontal [ ng ]}{MAL 2 ( C ) frontal [ ng ] / cyclophilin B ( C ) frontal [ ng ]}$$

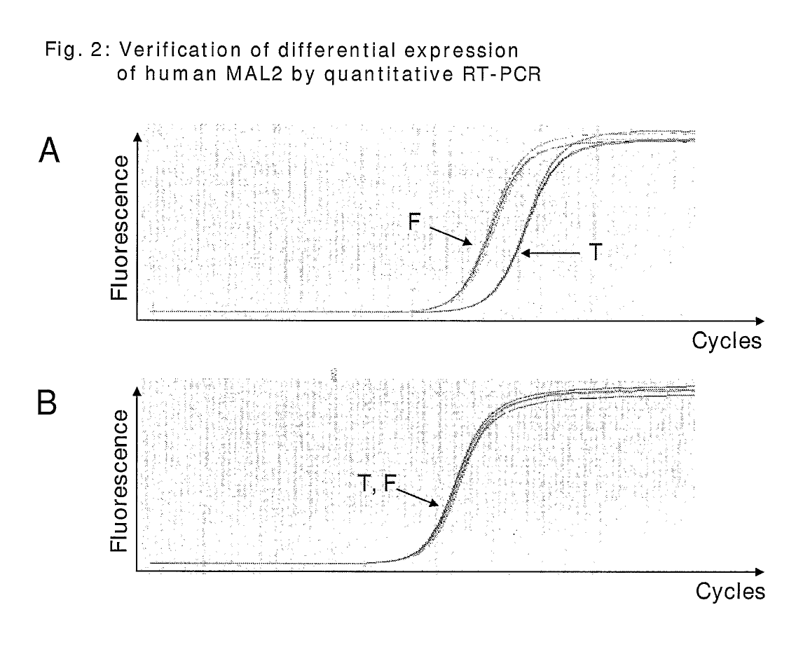

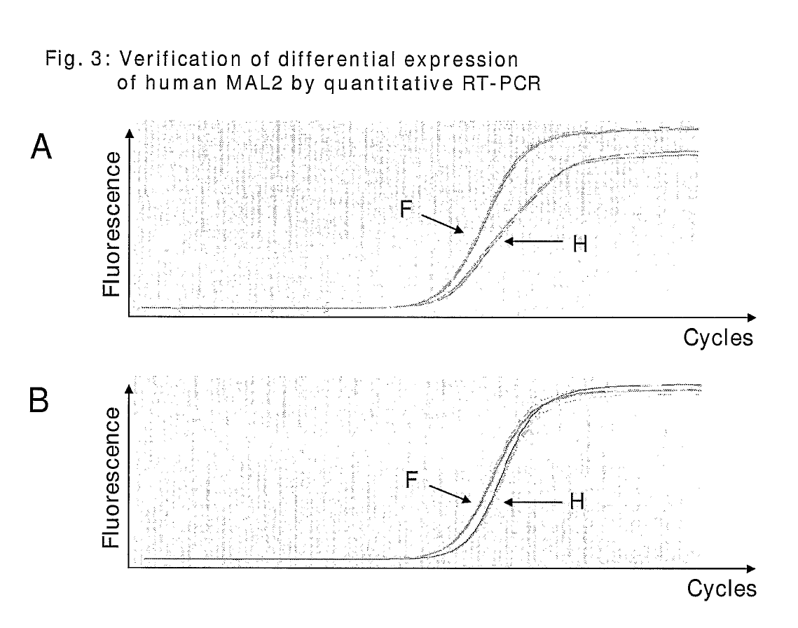

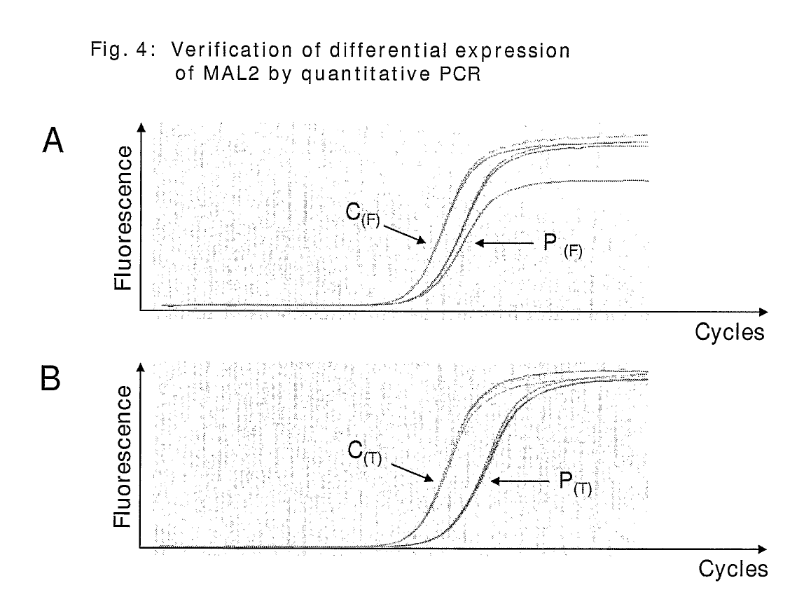

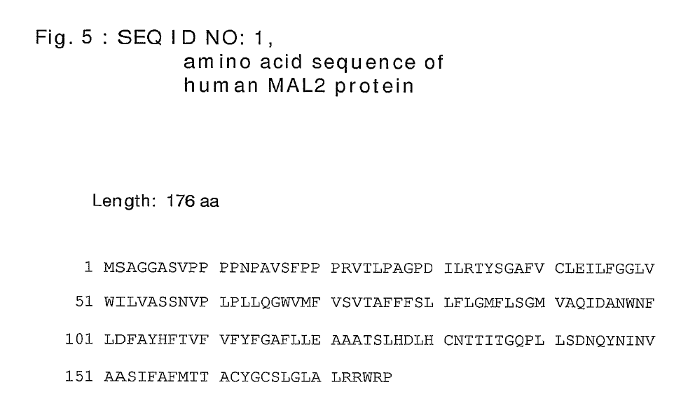

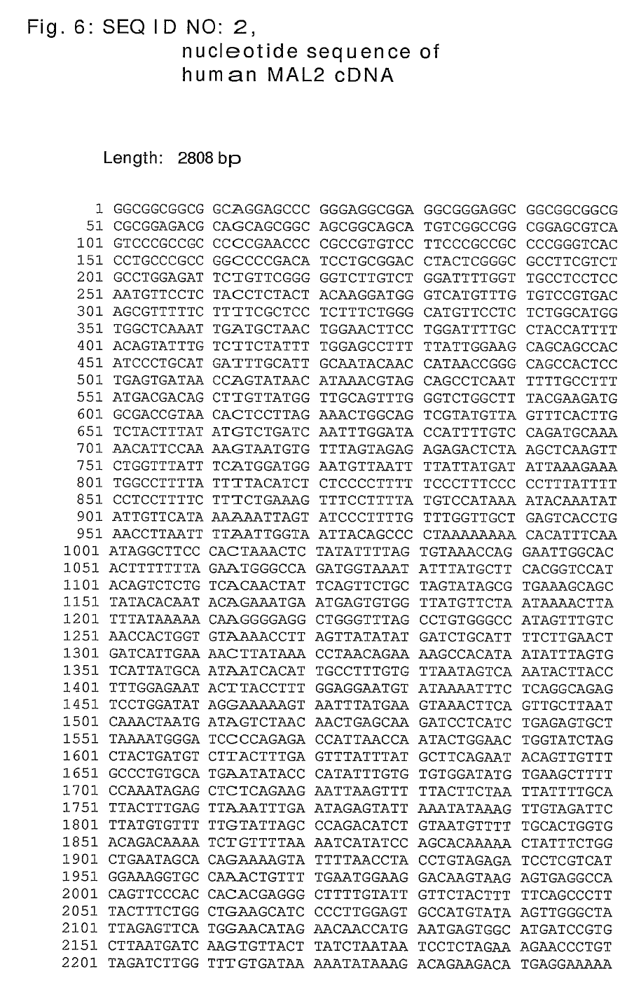

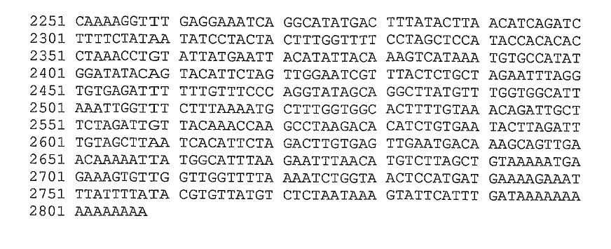

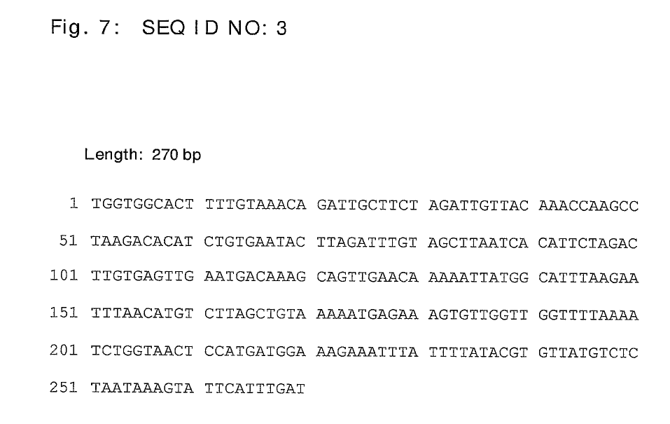

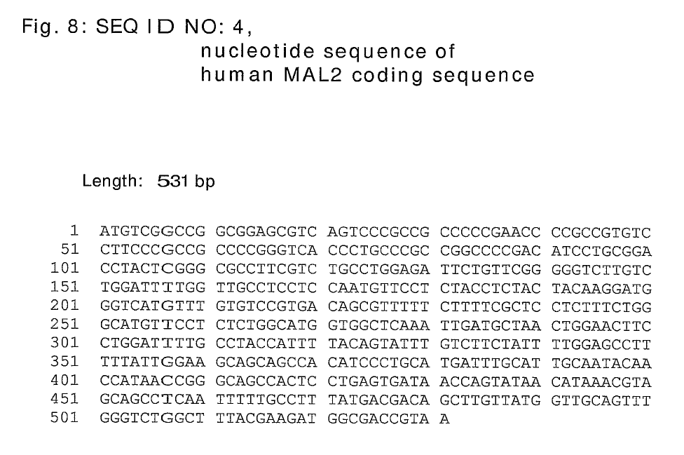

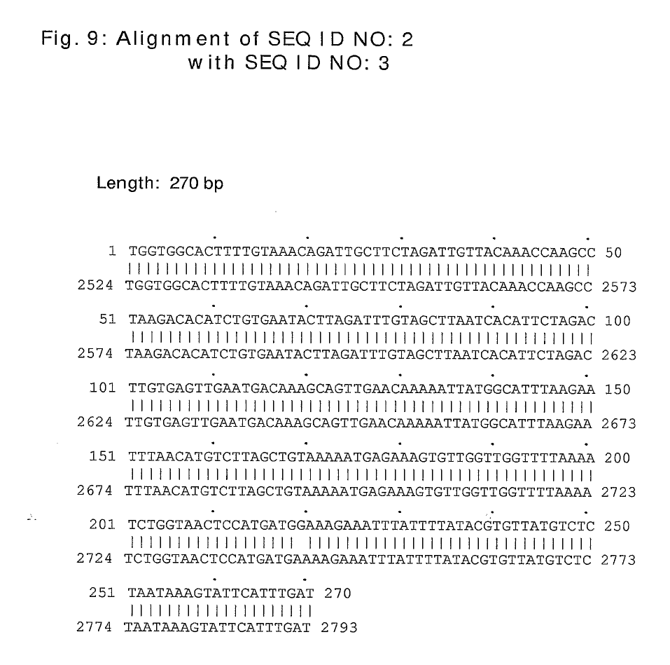

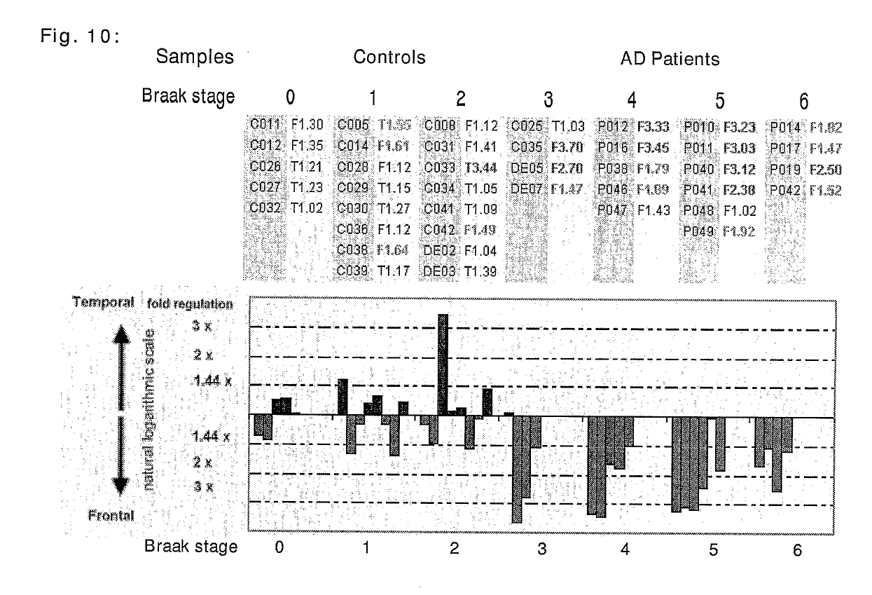

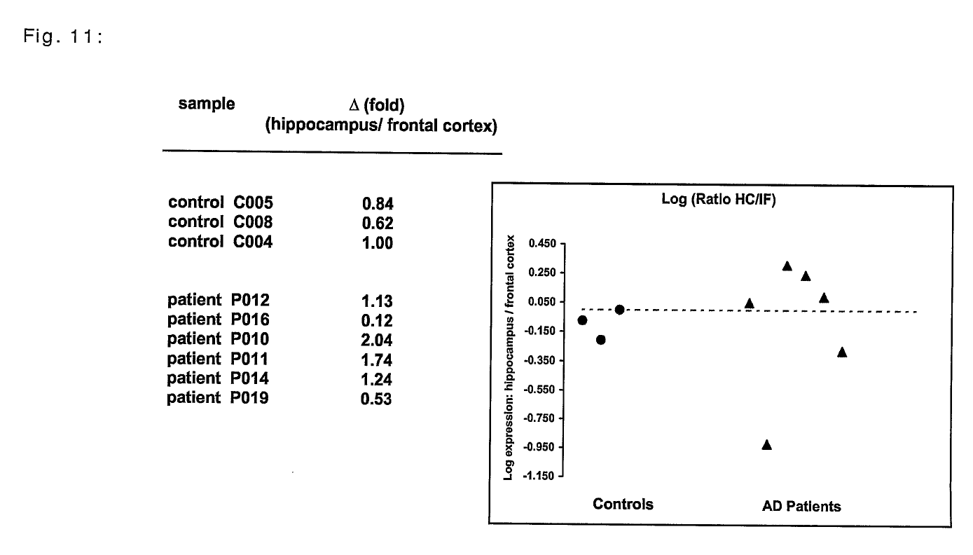

In a third step, the set of reference standard genes was analyzed in parallel to determine the mean average value of the AD patient to control person temporal cortex ratios, of the AD patient to control person frontal cortex ratios, and of the temporal to frontal cortex ratios, and of the hippocampal to frontal cortex ratios of AD patients and of control persons, respectively, of expression levels of the reference standard genes for each individual brain sample. As cyclophilin B was analyzed in step 2 and step 3, and the ratio from one gene to another gene remained constant in different runs, it was possible to normalize the values for the gene coding for MAL2 protein to the mean average value of the set of reference standard genes instead of normalizing to one single gene alone. The calculation was performed by dividing the respective ratios shown above by the deviation of cyclophilin B from the mean value of all housekeeping genes. The results of such quantitative RT-PCR analysis for the gene coding for MAL2 protein are shown in FIGS. 2, 3, 4 and 10 and 11.

2) Comparison of the mRNA Expression Between Controls and AD Patients.

For this analysis it was proven that absolute values of real-time quantitative PCR (Lightcycler method) between different experiments at different time points are consistent enough to be used for qualitative comparisons without usage of calibrators. Cyclophilin was used as a standard for normalization in any of the qPCR experiments for more than 100 tissues. Between others it was found to be the most consistently expressed housekeeping gene in our normalization experiments. Therefore a proof of concept was done by using values that were generated for cyclophilin.

First analysis used cyclophilin values from qPCR experiments of frontal cortex and inferior temporal cortex tissues from three different donors. From each tissue the same cDNA preparation was used in all analyzed experiments. Within this analysis no normal distribution of values was achieved due to small number of data. Therefore the method of median and its 98%-confidence level was applied. This analysis revealed a middle deviation of 8.7% from the median for comparison of absolute values and a middle deviation of 6.6% from the median for relative comparison.

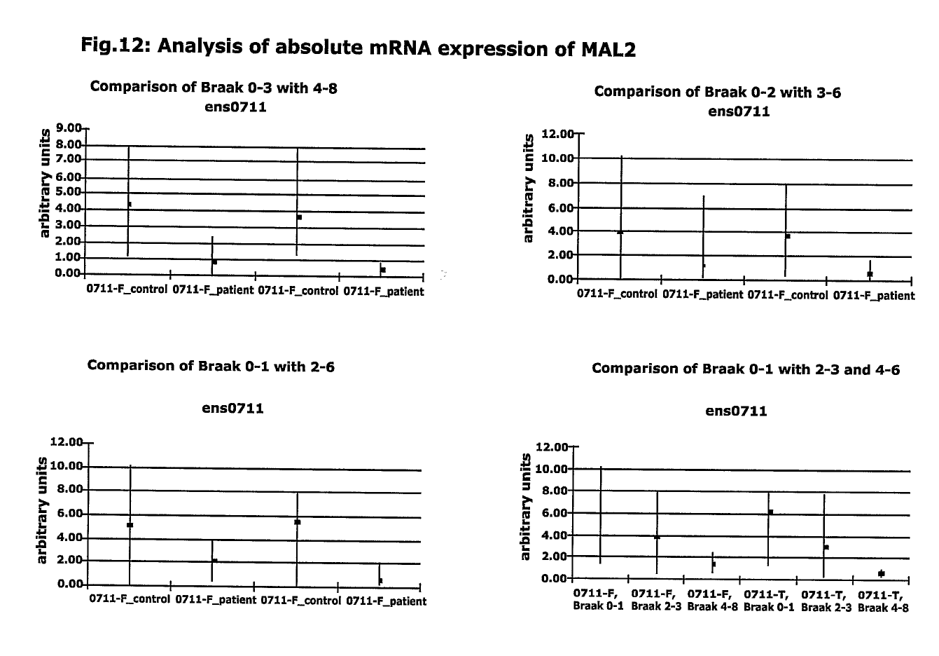

Second analysis used cyclophilin values from qPCR experiments of frontal cortex and inferior temporal cortex tissues from two different donors each, but different cDNA preparations from different time points were used. This analysis revealed a middle deviation of 29.2% from the median for comparison of absolute values and a middle deviation of 17.6% from the median for relative comparison. From this analysis it was concluded, that absolute values from qPCR experiments can be used, but the middle deviation from median should be taken into further considerations. A detailed analysis of absolute values for MAL2 was performed. Therefore, absolute levels of MAL2 were used after relative normalization with cyclophilin. The median as well as the 98%-confidence level was calculated for the control group (Braak 0-Braak 3) and the patient group (Braak 4-Braak 6), respectively. The same analysis was done redefining the control group (Braak 0-Braak 2) and the patient group (Braak 3-Braak 6) as well as redefining the control group (Braak 0-Braak 1) and the patient group (Braak 2-Braak 6). The latter analysis was aimed to identify early onset of mRNA expression differences between controls and AD patients. In another view of this analysis, three groups comprising Braak stages 0-1, Braak stages 2-3, and Braak stages 4-6, respectively, were compared to each other in order to identify tendencies of gene expression regulation as well as early onset differences. Said analysis as described above is shown in FIG. 12.

(v) Immunoblotting:

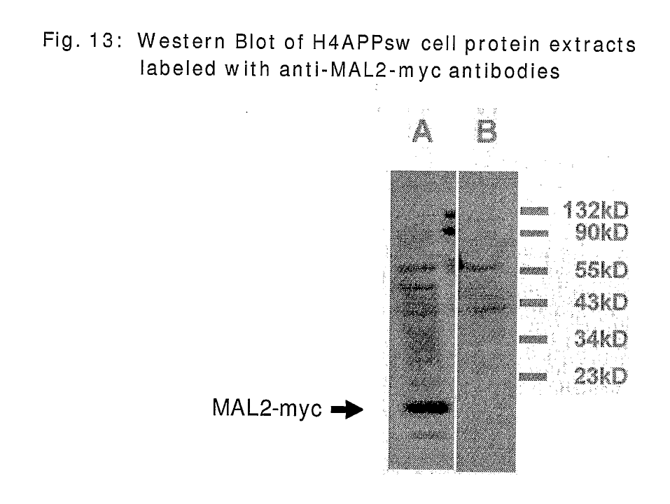

Total protein extract was obtained from H4APPsw cells expressing MAL2-myc by homogenization in 1 ml RIPA buffer (150 mM sodium chloride, 50 mM tris-HCl, pH7.4, 1 mM ethylenediamine-tetraacetic acid, 1 mM phenylmethylsulfonyl flouride, 1% Triton X-100, 1% sodium deoxycholic acid, 1% sodium dodecylsulfate, 5 μg/ml of aprotinin, 5 μg/ml of leupeptin) on ice. After centrifuging twice for 5 min at 3000 rpm at 4° C., the supernatant was diluted five-fold in SDS-loading buffer. Aliquots of 12 μl of the diluted sample were resolved by SDS-PAGE (8% polyacrylamide) and transferred to PVDF Western Blotting membranes (Boehringer Mannheim). The blots were probed with rabbit polyclonal anti-myc antibodies (MBL, 1:1000) followed by horseradish peroxidase-coupled goat anti-rabbit IgG antiserum (Santa Cruz sc-2030, diluted 1:5000) and developed with the ECL chemoluminescence detection kit (Amersham Pharmacia) (FIG. 13).

(vi) Immunofluorescence Analysis (IF):

For the immunofluorescence staining of MAL2 protein in cells, a human neuroglioma cell line was used (H4 cells) which stably expresses the human APP695 isoform carrying the Swedish mutation (K670N, M671L) (H4APPsw cells).

The H4APPsw cells were transduced with a pFB-Neo vector (Stratagene, #217561) containing the coding sequence of MAL2 (MAL2 cds) (SEQ ID NO:4, 531 bp) and a myc-tag (pFB-Neo-MAL2cds-myc, MAL2-myc vector, 7135 bp, EcoRI/XhoI) under the control of a strong CMV promotor. For the generation of the MAL2-myc vector, the MAL2cds-myc sequence was introduced into the EcoRI/XhoI restriction sites of the multiple cloning site (MCS) of the pFB-Neo vector. For transduction of the H4APPsw cells with the MAL2-myc vector the retroviral expression system ViraPort from Stratagene was used.

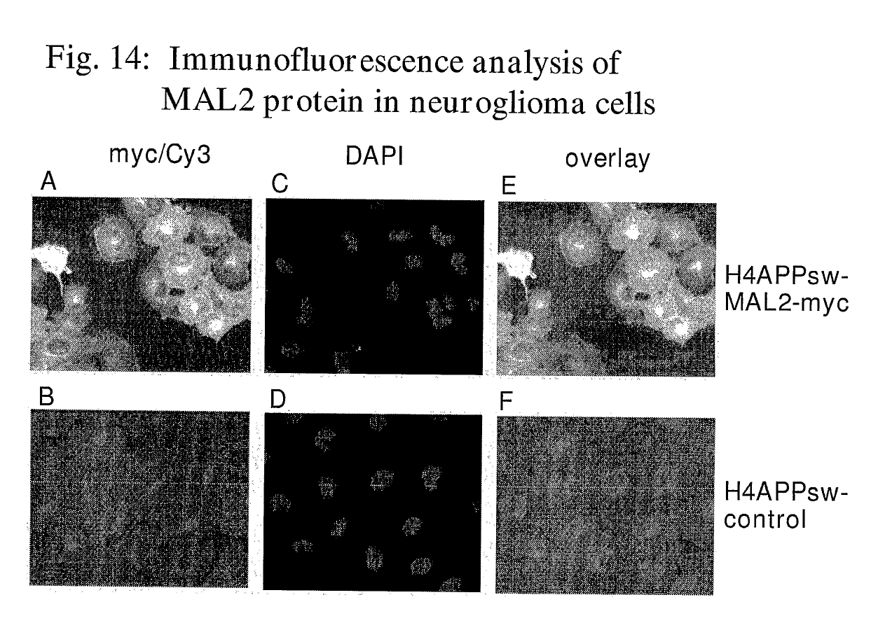

The myc-tagged MAL2 over-expressing cells (H4APPsw-MAL2cds-myc) were seeded onto glass cover slips in a 24 well plate (Nunc, Roskilde, Denmark; #143982) at a density of 5×10⁴ cells and incubated at 37° C. at 5% CO₂ over night. To fix the cells onto the cover slip, medium was removed and chilled methanol (−20° C.) was added. After an incubation period of 15 minutes at −20° C., methanol was removed and the fixed cells were blocked for 1 hour in blocking solution (200 μl PBS/5% BSA/3% goat serum) at room temperature. The first antibody (polyclonal anti-myc antibody, rabbit, 1:5000, MBL) and DAPI (DNA-stain, 0.05 μg/ml, 1:1000) in PBS/1% goat serum was added and incubated for 1 hour at room temperature. After removing the first antibody, the fixed cells were washed 3 times with PBS for 5 minutes. The second antibody (Cy3-conjugated anti-rabbit antibody, 1:1000, Amersham Pharmacia, Germany) was applied in blocking solution and incubated for 1 hour at room temperature. The cells were washed 3 times in PBS for 5 minutes. Coverslips were mounted onto microscope slides using Permafluor (Beckman Coulter) and stored over night at 4° C. to harden the mounting media. Cells were visualized using microscopic dark field epifluorescence and bright field phase contrast illumination conditions (IX81, Olympus Optical). Microscopic images (FIG. 14) were digitally captured with a PCO SensiCam and analysed using the appropriate software (AnalySiS, Olympus Optical).

(vii) Immunohistochemistry:

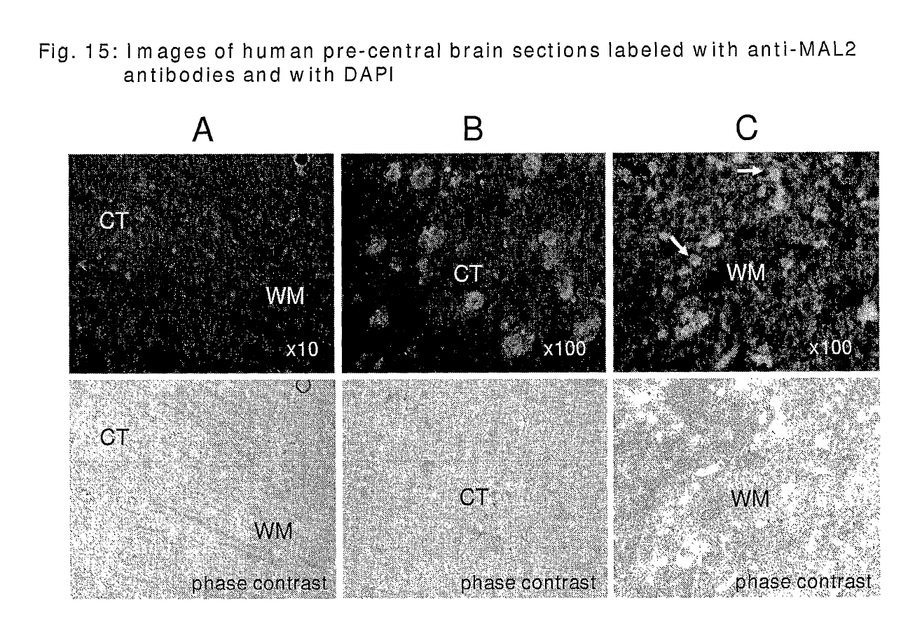

For immunofluorescence staining of MAL2 in human brain, frozen sections were prepared with a cryostat (Leica CM3050S) from post-mortem frontal cortex of a donor person and fixed in 4% PFA 20 min at room temperature. After washing in PBS, the sections were pre-incubated with blocking buffer (10% normal goat serum, 0.2% Triton X-100 in PBS) for 30 min and then incubated with affinity-purified rabbit polyclonal anti-MAL2 antisera (1:60 diluted in blocking buffer; Davids Biotechnology, Regensburg) and with a mouse monoclonal anti-CNPase antibody (C5P22; Sigma) overnight at 4° C. After rinsing three times in 0.1% Triton X-100/PBS, the sections were incubated with FITC-conjugated goat anti-rabbit IgG antisera (1:150 diluted in 1% BSA/PBS) and with a Cy3-conjugated goat anti-mouse IgG antiserum (1:600) for 2 hours at room temperature and then again washed in PBS. Staining of the nuclei was performed by incubation of the sections with 5 μM DAPI in PBS for 3 min (blue signal). In order to block the autofluoresence of lipofuscin in human brain, the sections were treated with 1% Sudan Black B in 70% ethanol for 2-10 min at room temperature and then sequentially dipped in 70% ethanol, destined water and PBS. The sections were coverslipped with ‘Vectrashield’ mounting medium (Vector Laboratories, Burlingame, Calif.) and observed under an inverted microscope (IX81, Olympus Optical). The digital images were captured with the appropriate software (AnalySiS, Olympus Optical) (FIG. 15).

1. A method of diagnosing or prognosticating Alzheimer's disease in a subject, or determining whether a subject is at increased risk of developing said disease, comprising determining a level and/or an activity of
(i) a transcription product of the gene coding for MAL2 protein, and/or
(ii) a translation product of the gene coding for MAL2 protein, and/or
(iii) a fragment, or derivative, or variant of said transcription or translation product,
in a sample obtained from said subject and comparing said level and/or said activity of said transcription product and/or said translation product to a reference value representing a known disease status and/or to a reference value representing a known health status, and said level and/or said activity is varied compared to a reference value representing a known health status, and/or is similar or equal to a reference value representing a known disease status, thereby diagnosing or prognosticating Alzheimer's disease in said subject, or determining whether said subject is at increased risk of developing said disease.

2. A kit for diagnosing or prognosticating a neurodegenerative disease in a subject, or determining the propensity or predisposition of a subject to develop such a disease, said kit comprising at least one reagent which is selected from the group consisting of
(i) reagents that detect a transcription product of the gene coding for MAL2 protein and
(ii) reagents that detect a translation product of the gene coding for MAL2 protein, whereby the diagnosis or prognosis or determination of the propensity or predisposition to develop said neurodegenerative disease is determined by the steps of:
(a) detecting in a sample obtained from said subject a level, or an activity, or both said level and said activity of a transcription product and/or of a translation product of a gene coding for MAL2, and
(b) comparing said level or activity, or both said level and said activity of a transcription product and/or of a translation product of a gene coding for MAL2 to a reference value representing a known health status and/or to a reference value representing a known disease status, and said level, or activity, or both said level and said activity, of said transcription product and/or said translation product of a gene coding for MAL2 is varied compared to a reference value representing a known health status, and/or is similar or equal to a reference value representing a known disease status.

3. A modulator of an activity and/or of a level of at least one substance which is selected from the group consisting of
(i) a gene coding for MAL2 protein, and/of
(ii) a transcription product of the gene coding for MAL2 protein,
(iii) a translation product of the gene coding for MAL2 protein, and
(iv) a fragment, or derivative, or variant of (i) to (iii).

4. A recombinant, non-human animal comprising a non-native gene sequence coding for MAL2 or a fragment, or a derivative, or a variant thereof, said animal being obtainable by:
(i) providing a gene targeting construct comprising said gene sequence and a selectable marker sequence,
(ii) introducing said targeting construct into a stem cell of a non-human animal,
(iii) introducing said non-human animal stem cell into a non-human embryo,
(iv) transplanting said embryo into a pseudopregnant non-human animal,
(v) allowing said embryo to develop to term,
(vi) identifying a genetically altered non-human animal whose genome comprises a modification of said gene sequence in both alleles, and
(vii) breeding the genetically altered non-human animal of step (vi) to obtain a genetically altered non-human animal whose genome comprises a modification of said endogenous gene, wherein said modification results in said non-human animal exhibiting a predisposition to developing symptoms of a neurodegenerative disease or related diseases or disorders.

5. A method of diagnosing or therapy for treating neurodegenerative diseases, comprising screening, testing, and validating compounds, agents, and modulators using the genetically altered non-human animal of claim 4.

6. A method for screening for a modulator of neurodegenerative diseases or related diseases or disorders of one or more substances selected from the group consisting of
(i) a gene coding for MAL2 protein,
(ii) a transcription product of the gene coding for MAL2 protein,
(iii) a translation product of the gene coding for MAL2 protein, and
(iv) a fragment, or derivative, or variant of (i) to (iii),
said method comprising:
(a) contacting a cell with a test compound;
(b) measuring the activity and/or level of one or more substances recited in (i) to (iv);
(c) measuring the activity and/or level of one or more substances recited in (i) to (iv) in a control cell not contacted with said test compound; and comparing the levels and/or activities of the substance in the cells of step (b) and (c), wherein an alteration in the activity and/or level of substances in the contacted cells indicates that the test compound is a modulator of said diseases or disorders.

7. A method of screening for a modulator of neurodegenerative diseases or related diseases or disorders of one or more substances selected from the group consisting of
(i) the gene coding for MAL2 protein,
(ii) a transcription product of the gene coding for MAL2 protein,
(iii) a translation product of the gene coding for MAL2 protein, and
(iv) a fragment, or derivative, or variant of (i) to (iii),
said method comprising:
(a) administering a test compound to a test animal which is predisposed to developing or has already developed symptoms of a neurodegenerative disease or related diseases or disorders in respect of the substances recited in (i) to (iv);
(b) measuring the activity and/or level of one or more substances recited in (i) to (iv);
(c) measuring the activity and/or level of one or more substances recited in (i) or (iv) in a matched control animal which is predisposed to developing or has already developed symptoms of a neurodegenerative disease or related diseases or disorders in respect to the substances recited in (i) to (iv) and to which animal no such test compound has been administered; and
(d) comparing the activity and/or level of the substance in the animals of steps (b) and (c), wherein an alteration in the activity and/or level of substances in the test animal indicates that the test compound is a modulator of said diseases or disorders.

8. The method according to claim 7 wherein said test animal and/or said control animal is a recombinant animal which expresses MAL2, or a fragment, or a derivative, or a variant thereof, under the control of a transcriptional control element which is not the native MAL2 gene transcriptional control element.

9. An assay for testing a compound, or a plurality of compounds to determine the degree of binding of said compounds to MAL2 protein, or to a fragment, or derivative, or variant thereof, said assay comprising:
(i) adding a liquid suspension of said MAL2 protein, or a fragment, or derivative, or variant thereof, to a plurality of containers;
(ii) adding a detectable compound or a plurality of detectable compounds to be screened for said binding to said plurality of containers;
(iii) incubating said MAL2 protein, or said fragment, or derivative, or variant thereof, and said detectable compound or detectable compounds;
(iv) measuring amounts of detectable compound or compounds associated with said MAL2 protein, or with said fragment, or derivative, or variant thereof; and
determining the degree of binding by one or more of said compounds to said MAL2 protein, or said fragment, or derivative, or variant thereof.

10. The method of claim 1, comprising determining a level and/or activity of a protein molecule of SEQ ID NO:1, said protein molecule being a translation product of the gene coding for MAL2, or a fragment, or derivative, or variant thereof.

11. The method of claim 10, wherein said screening is for identification of a modulator of a protein molecule of SEQ ID NO: 1, said protein molecule being a translation product of the gene coding for MAL2, or a fragment, or derivative, or variant thereof.

12. A method for detecting the pathological state of a cell in a sample obtained from a subject, comprising immunocytochemical staining of said cell with an antibody specifically immunoreactive with an immunogen, wherein said immunogen is a translation product of a gene coding for MAL2, SEQ ID NO: 1, or a fragment, or derivative, or variant thereof, wherein an altered degree of staining, or an altered staining pattern in said cell compared to a cell representing a known health status indicates a pathological state of said cell which relates to a neurodegenerative disease.

13. The kit of claim 2, wherein said neurodegenerative disease is Alzheimer's disease.

14. The kit of claim 2, wherein said translation product is a protein molecule of SEQ ID NO: 1, said protein molecule being a translation product of the gene coding for MAL2, or a fragment, or derivative, or variant thereof.

15. The method of claim 5, wherein said neurodegenerative disease is Alzheimer's disease.

16. The method of claim 6, wherein said neurodegenerative disease is Alzheimer's disease.

17. The method of claim 7, wherein said neurodegenerative disease is Alzheimer's disease.

18. The assay of claim 9, wherein said detectable compound is fluorescently labeled.

19. The method of claim 12, wherein said neurodegenerative disease is Alzheimer's disease.
